(19) United States
(12) Patent Application Publication
(10) Pub. No.: US 2023/0340600 A1
(43) Pub. Date: Oct. 26, 2023

(54) Synchronized Cell-Based Compositions

(71) Applicant: NeuroGX LLC, New York, NY (US)

(72) Inventors: Florin Valentin Chirila, Morgantown, WV (US); Daniel L. Alkon, Chevy Chase, MO (US)

(73) Assignee: NeuroGX LLC, New York, NY (US)

(21) Appl. No.: 18/295,893

(22) Filed: Apr. 5, 2023

Related U.S. Application Data
(63) Continuation of application No. 16/434,362, filed on Jun. 7, 2019, now abandoned.
Application No. 16/434,362 is a continuation-in-part of application No. PCT/US18/64322, filed on Dec. 6, 2018.
(60) Provisional application No. 62/596,588, filed on Dec. 8, 2017.

(51) Int. Cl. C12Q 1/6883

(52) U.S. Cl. CPC C12Q 1/6883; C12Q 2600/158

(57) ABSTRACT

This invention provides a method for determining whether a human subject is afflicted with AD or non-ADD when the subject is suspected of being afflicted with AD or non-ADD, comprising the steps of (a) synchronizing a population of suitable cells derived from the subject; and (b) in the resulting synchronized cell population, measuring the expression level of a gene known to be differentially expressed between corresponding synchronized cells derived from AD patients and those derived from non-ADD patients, whereby (i) the subject is afflicted with AD if the expression level measured in step (b) is consistent with that gene's expression level in corresponding synchronized cells derived from AD patients, and (ii) the subject is afflicted with non-ADD if the expression level measured in step (b) is consistent with that gene's expression level in corresponding synchronized cells derived from non-ADD patients. This invention also provides diagnostic methods based on NDS patient gene expression levels. Finally, this invention provides methods for treating a subject afflicted with AD comprising administering a therapeutically effective amount of an agent known to favorably affect the expression level of one or more genes whose expression levels correlate with Alzheimer's disease.

This application is a continuation-in-part of PCT International Application No. PCT/US2018/64322, filed Dec. 6, 2018, and claims the benefit of U.S. Provisional Application No. 62/596,588, filed Dec. 8, 2017, and PCT International Application No. PCT/US2018/64322, filed Dec. 6, 2018, the contents of both of which are incorporated herein by reference.

Throughout this application, various publications are cited. The disclosure of these publications is hereby incorporated by reference into this application to describe more fully the state of the art to which this invention pertains.

### BACKGROUND OF THE INVENTION

Alzheimer's disease (“AD”) has long been the subject of considerable efforts to develop accurate diagnostic methods, as well as therapeutic methods. Despite these efforts, there is an unmet need for methods of accurately diagnosing AD and differentiating it from non-Alzheimer's dementia (“non-ADD”). There is also an unmet need for effective methods of treating AD.

### SUMMARY OF THE INVENTION

This invention provides a method for determining whether a human subject is afflicted with AD or non-ADD when the subject is suspected of being afflicted with AD or non-ADD, comprising the steps of
(a) synchronizing a population of suitable cells derived from the subject; and
(b) in the resulting synchronized cell population, measuring the expression level of a gene known to be differentially expressed between corresponding synchronized cells derived from AD patients and those derived from non-ADD patients,
   whereby (i) the subject is afflicted with AD if the expression level measured in step (b) is consistent with that gene's expression level in corresponding synchronized cells derived from AD patients, and (ii) the subject is afflicted with non-ADD if the expression level measured in step (b) is consistent with that gene's expression level in corresponding synchronized cells derived from non-ADD patients.

This invention also provides a method for determining whether a human subject is afflicted with AD or non-ADD when the subject is suspected of being afflicted with AD or non-ADD, comprising the steps of
(a) synchronizing a population of cultured skin cell fibroblasts derived from the subject, wherein the synchronizing comprises culturing the fibroblasts to over-confluence and then starving the resulting over-confluent fibroblasts; and
(b) in the resulting synchronized fibroblast population, measuring the expression level of each of genes AC004057.1, AC092651.1, ACP6, ADAM20, ASXL2, C2CD5, CARNS1, FAM149B1, GLIS3-AS1, IL18R1, LINC01393, LZIC, MAP1LC3B2, NHLH1, NORAD, NPPA-AS1_3, OSMR-AS1, PAN3, PHBP8, PSMB9, RAB3IP, RDH16, RFESDP1, RPL5, SCG2, SDHD, SHISA5, SLC45A3, SNHG14, TTC26, URB2, USMG5, WASF2, ZCWPW2, ZNF444, and ZNF70, wherein measuring the expression level of each gene comprises measuring the number of its RNA transcripts per number of total transcripts,
   whereby (i) the subject is afflicted with AD if the expression levels measured in step (b) are consistent with the genes' expression levels in corresponding synchronized cells derived from AD patients, and (ii) the subject is afflicted with non-ADD if the expression levels measured in step (b) are consistent with the genes' expression levels in corresponding synchronized cells derived from non-ADD patients.

This invention further provides a method for determining whether a human subject is afflicted with AD or non-ADD when the subject is suspected of being afflicted with AD or non-ADD, comprising the steps of
(a) synchronizing a population of cultured immortalized B lymphocytes derived from the subject, wherein the synchronizing comprises culturing the lymphocytes to over-confluence and then starving the resulting over-confluent lymphocytes; and
(b) in the resulting synchronized lymphocyte population, measuring the expression level of each of genes AC004057.1, AC092651.1, ACP6, ADAM20, ASXL2, C2CD5, CARNS1, FAM149B1, GLIS3-AS1, IL18R1, LINC01393, LZIC, MAP1LC3B2, NHLH1, NORAD, NPPA-AS1_3, OSMR-AS1, PAN3, PHBP8, PSMB9, RAB3IP, RDH16, RFESDP1, RPL5, SCG2, SDHD, SHISA5, SLC45A3, SNHG14, TTC26, URB2, USMG5, WASF2, ZCWPW2, ZNF444, and ZNF70, wherein measuring the expression level of each gene comprises measuring the number of its RNA transcripts per number of total transcripts,
   whereby (i) the subject is afflicted with AD if the expression levels measured in step (b) are consistent with the genes' expression levels in corresponding synchronized cells derived from AD patients, and (ii) the subject is afflicted with non-ADD if the expression levels measured in step (b) are consistent with the genes' expression levels in corresponding synchronized cells derived from non-ADD patients.

This invention provides a method for determining whether a human subject is afflicted with AD or is a NDS when the subject is suspected of being afflicted with AD, comprising the steps of
(a) synchronizing a population of suitable cells derived from the subject; and
(b) in the resulting synchronized cell population, measuring the expression level of a gene known to be differentially expressed between corresponding synchronized cells derived from AD patients and those derived from NDS patients,
   whereby (i) the subject is afflicted with AD if the expression level measured in step (b) is consistent with that gene's expression level in corresponding synchronized cells derived from AD patients, and (ii) the subject is a NDS if the expression level measured in step (b) is consistent with that gene's expression level in corresponding synchronized cells derived from NDS patients.

This invention also provides a method for determining whether a human subject is afflicted with AD or is a NDS when the subject is not suspected of being afflicted with AD, comprising the steps of
(a) synchronizing a population of suitable cells derived from the subject; and
(b) in the resulting synchronized cell population, measuring the expression level of a gene known to be differentially expressed between corresponding synchronized cells derived from AD patients and those derived from NDS patients,
   whereby (i) the subject is afflicted with AD if the expression level measured in step (b) is consistent with that gene's expression level in corresponding synchronized cells derived from AD patients, and (ii) the subject is a NDS if the expression level measured in step (b) is consistent with that gene's expression level in corresponding synchronized cells derived from NDS patients.

This invention further provides a method for treating a human subject afflicted with Alzheimer's disease comprising administering to the subject a therapeutically effective amount of an agent known to favorably affect the expression level of one or more genes whose expression levels correlate with Alzheimer's disease.

Finally, this invention provides methods for treating a human subject afflicted with Alzheimer's disease comprising administering to the subject a therapeutically effective amount of carfilzomib, bortezomib, bumetanide, furosemide or torsemide.

### BRIEF DESCRIPTION OF THE FIGURES

FIG. 1

This Figure, based on a first study (“Study 1”), shows statistically significant genes when comparing the AD group with the Non-ADD group. Study 1 revealed that there are 2103 statistically significant genes for a P level less than 0.1; 1099 statistically significant genes for a P level less than 0.05; 285 statistically significant genes for a P level less than 0.01; and 6 statistically significant genes for a P level less or equal than 0.001.

FIG. 2

This Figure, based on Study 1, shows the top 6 statistically significant genes (P<=0.001) for the 6 AD and 2 Non-ADD cases. Squares represent the AD population while circles represent the Non-ADD population.

FIG. 3A

This Figure, based on Study 1, shows an example of the top 10 statistically significant genes (P<=0.01). Specifically, in this Figure, Raw TPM (transcripts per million) data show with squares the AD population and with circles the Non-ADD population.

FIG. 3B

This Figure, based on Study 1, shows an example of the top 10 statistically significant genes (P<=0.01). Specifically, in this Figure, Average TPM data show with squares the AD population and with circles the Non-ADD population. Error bars are standard deviations.

FIG. 3C

This Figure, based on Study 1, shows an example of the top 10 statistically significant genes (P<=0.01). Specifically, in this Figure, Percent change (% Ch) in gene expression is shown when comparing the AD with control (Non-ADD), i.e., 100*(AD-Non-ADD)/Non-ADD.

FIG. 4A

This Figure, based on Study 1, shows genes ranked 11 to 20 at the statistical significance of 1% overlap probability (P<=0.01). Specifically, in this Figure, Raw TPM data show with squares the AD population and with circles the Non-ADD population.

FIG. 4B

This Figure, based on Study 1, shows genes ranked 11 to 20 at the statistical significance of 1% overlap probability (P<=0.01). Specifically, in this Figure, Average TPM data show with squares the AD population and with circles the Non-ADD population. Error bars are standard deviations.

FIG. 4C

This Figure, based on Study 1, shows genes ranked 11 to 20 at the statistical significance of 1% overlap probability (P<=0.01). Specifically, in this Figure, Percent change (% Ch) in gene expression is shown when comparing the AD with control (Non-ADD), i.e., 100*(AD-Non-ADD)/Non-ADD.

FIG. 5A

This Figure, based on Study 1, shows genes ranked 21 to 30 at the statistical significance of 1% overlap probability (P<=0.01). Specifically, in this Figure, Raw TPM data show with squares the AD population and with circles the Non-ADD population.

FIG. 5B

This Figure, based on Study 1, shows genes ranked 21 to 30 at the statistical significance of 1% overlap probability (P<=0.01). Specifically, in this Figure, Average TPM data show with squares the AD population and with circles the Non-ADD population. Error bars are standard deviations.

FIG. 5C

This Figure, based on Study 1, shows genes ranked 21 to 30 at the statistical significance of 1% overlap probability (P<=0.01). Specifically, in this Figure, Percent change (% Ch) in gene expression is shown when comparing the AD with control (Non-ADD), i.e., 100*(AD-Non-ADD)/Non-ADD.

FIG. 6A

This Figure, based on Study 1, shows genes ranked 31 to 40 at the statistical significance of 1% overlap probability (P<=0.01). Specifically, in this Figure, Raw TPM data show with squares the AD population and with circles the Non-ADD population.

FIG. 6B

This Figure, based on Study 1, shows genes ranked 31 to 40 at the statistical significance of 1% overlap probability (P<=0.01). Specifically, in this Figure, Average TPM data show with squares the AD population and with circles the Non-ADD population. Error bars are standard deviations.

FIG. 6C

This Figure, based on Study 1, shows genes ranked 31 to 40 at the statistical significance of 1% overlap probability (P<=0.01). Specifically, in this Figure, Percent change (% Ch) in gene expression is shown when comparing the AD with control (Non-ADD), i.e., 100*(AD-Non-ADD)/Non-ADD.

FIG. 7

This Figure, based on Study 1, shows the percent change (% Ch) in gene expression for the top 40 genes.

FIG. 8

This Figure, based on a second study (“Study 2”), shows the number of statistically significant differentially expressed genes for the training set (first cylinders-lighter shading) versus the number of statistically significant differentially expressed genes for the validation set (second cylinders-darker shading) for different levels of statistical significance P<0.001, 0.01, 0.05, and 0.10.

FIG. 9A

This Figure, based on Study 2, shows gene networks for PAN3.

FIG. 9B

This Figure, based on Study 2, shows gene networks for PSMB9.

FIG. 9C

This Figure, based on Study 2, shows gene networks for TTC26.

FIG. 9D

This Figure, based on Study 2, shows gene networks for ZNF444.

FIG. 9E

This Figure, based on Study 2, shows gene networks for NHLH1.

FIG. 9F

This Figure, based on Study 2, shows gene networks for URB2.

FIG. 9G

This Figure, based on Study 2, shows gene networks for ADAM20.

FIG. 10A

This Figure, based on Study 2, shows network measures for cross-validated genes with respect to the number of edges.

FIG. 10B

This Figure, based on Study 2, shows network measures for cross-validated genes with respect to the average node degree.

FIG. 10C

This Figure, based on Study 2, shows network measures for cross-validated genes with respect to the average local clustering coefficient.

FIG. 11

This Figure, based on Study 2, compares Non-ADD (n=3) with Non-Demented Controls (NDC; n=5), and shows the number of differentially expressed genes in the Non-ADD population when compared with the NDC population.

FIG. 12

This Figure shows the number of statistically significant dysregulated genes when comparing AD (n=6) and Non-Demented Controls (n=5). Gene numbers are shown for P<0.0001, P<0.001, P<0.01, and P<0.05.

FIG. 13

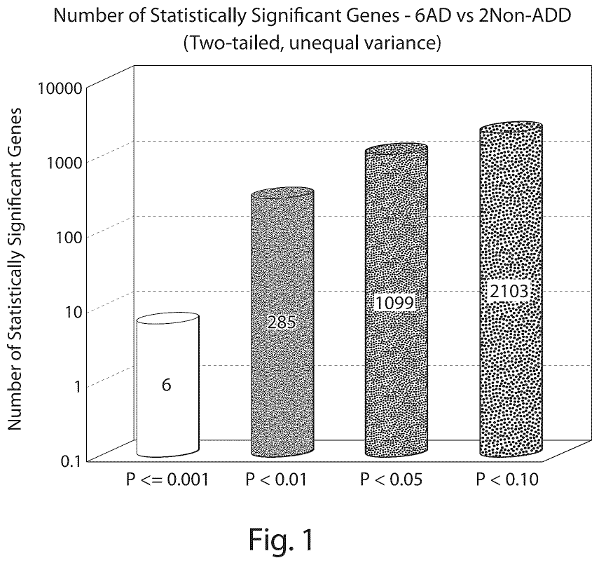

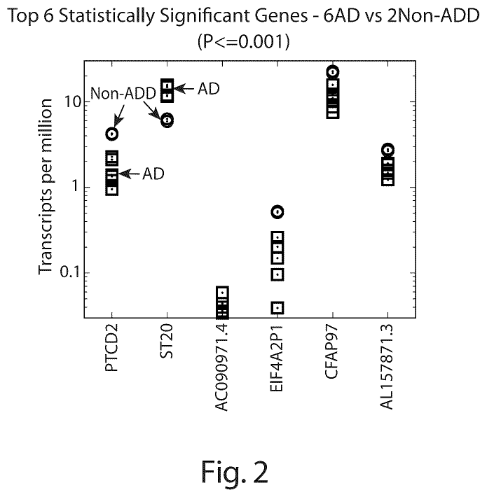

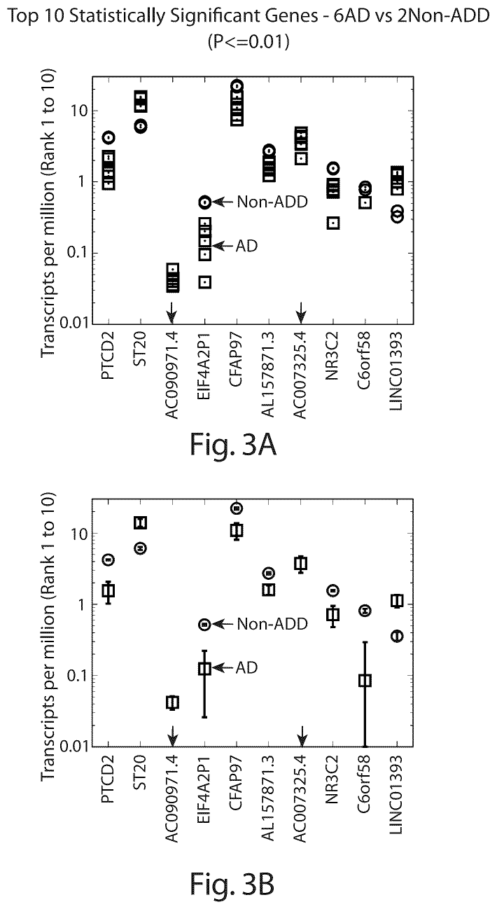

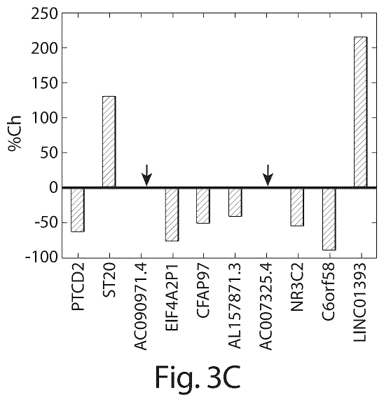

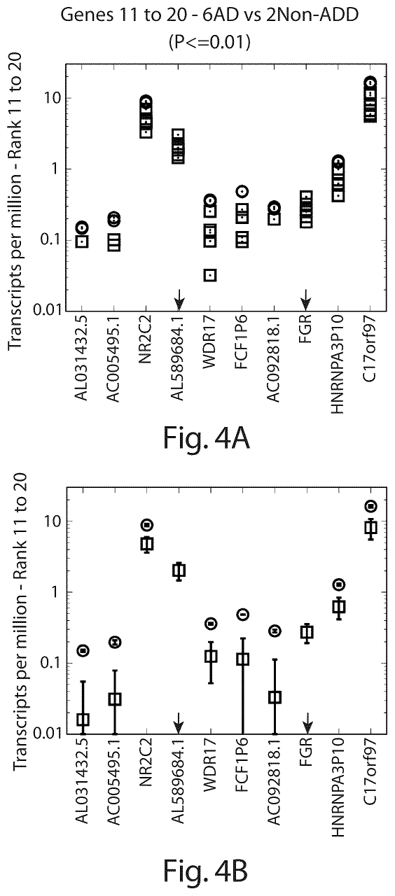

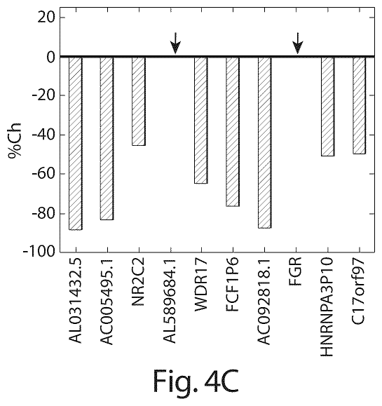

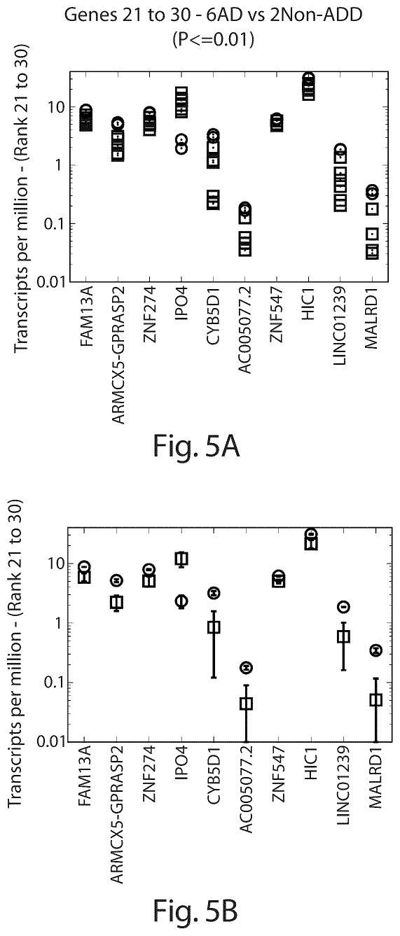

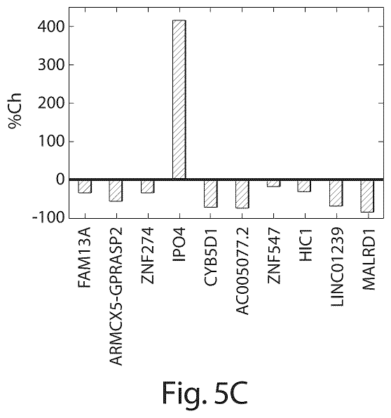

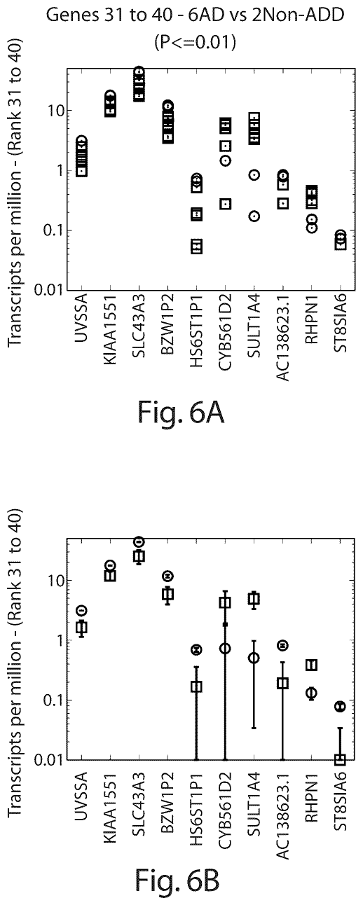

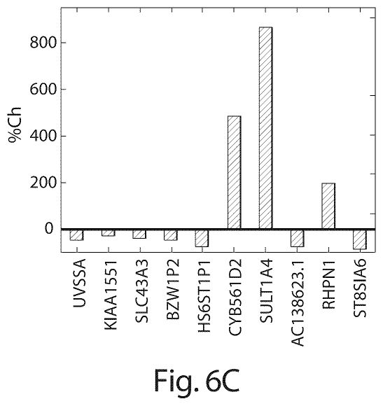

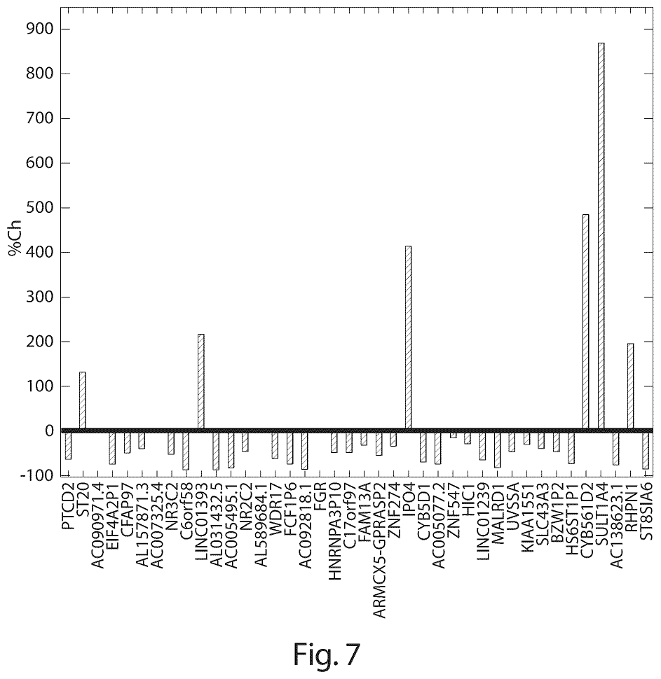

This Figure shows predicted gene expression profile changes with Alzheimer's disease severity. The current gene expression dysregulations for 26 cross-validated genes were ranked according to the percent change of the AD group FPKM (fragments per kilobase million) when compared with the FPKM for the Non-ADD group (blue). The gene CARNS1 has the largest percent change while the gene C2CD5 has the lowest percent change. The cylinders above zero indicate up-regulation for that specific gene while the cylinders below zero indicate down-regulation. The blue cylinders indicate the current data, which were obtained from patients with high severity of AD/Non-ADD disease. The red, grey, and yellow cylinders represent our prediction of how the pattern of the 26 dysregulated genes would look like for lower severities, i.e., ½, ¼, and ⅛ of the current data, based on the assumption that disease severity linearly correlates with the FPKM percent change.

### DETAILED DESCRIPTION OF THE INVENTION

### Definitions

In this application, certain terms are used which shall have the meanings set forth as follows.

As used herein, “administer”, with respect to an agent, means to deliver the agent to a subject's body via any known method. Specific modes of administration include, without limitation, intravenous, oral, sublingual, transdermal, subcutaneous, intraperitoneal and intrathecal administration.

In addition, in this invention, the various agents can be formulated using one or more routinely used pharmaceutically acceptable carriers. Such carriers are well known to those skilled in the art. For example, oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Injectable drug delivery systems include, for example, solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs and can contain excipients such as PLGA and polycaprylactone.

As used herein, “Alzheimer's disease” means a concurrent affliction with the following three symptoms: (i) dementia; (ii) amyloid plaques; and (iii) neurofibrillary tangles. Dementia can be diagnosed during life. Cerebral amyloid plaques and neurofibrillary tangles can, for example, be diagnosed during autopsy. This definition of Alzheimer's disease is the one provided by the National Institute of Neurological Disorders and Stroke (NINDS) of the National Institutes of Health (NIH), and is known as the “gold standard.” All disease-afflicted subjects from whom samples were taken and studied, and for which data are presented herein, are autopsy-confirmed AD, non-ADD patients, and NDCs (who were hypervalidated because they were not demented at the time of biopsy collection).

As used herein, a gene's expression level is “consistent” with that gene's expression level in corresponding synchronized cells derived from AD patients if it is the same as, or close to, that expression level. For example, assume that gene X's TPM measure in synchronized cells derived from AD patients is 10 and its TPM measure is 100 in the same type of cells derived from non-ADD (or NDC) patients that are synchronized in the same way. A subject's gene X expression level would be consistent with gene X's AD expression level if it were, for example, below 50, below 40, below 30, below 20 or, ideally, 10 or lower.

As used herein, “culturing” lymphocytes is achieved, for example, by conducting the culturing at a temperature and in a growth factor milieu permissive of cell growth. In another embodiment, “culturing” lymphocytes is performed under conditions (e.g., those described herein for proliferation) that preserve lymphocyte viability. In one embodiment, the temperature, salinity and protein milieu permissive of cell growth is 37° C., RPMI 1640 Medium with 10% fetal bovine serum (“FBS”) and 1% penicillin (“PS”). In one embodiment of this invention, the lymphocyte-culturing step is performed for more than three hours. Preferably, the lymphocyte-culturing step is performed for more than six hours (e.g., overnight). B-lymphocyte can be cultured to over-confluence, i.e., high density/μl. The high density is determined as the plateau that is typically more then 90% in the growth curve. Then, the lymphocytes are starved overnight.

Methods for obtaining lymphocytes from a subject's blood are known, and include, for example, flow cytometry, Ficoll (a hydrophilic polysaccharide that separates layers of blood), and gradient centrifugation. Additionally, in the subject methods, the lymphocytes (e.g., B lymphocytes) can be used in immortalized or primary (i.e., non-immortalized) form. Methods for immortalizing lymphocytes (e.g., B lymphocytes) are known, and include, for example, treating the lymphocytes with Epstein-Barr virus (“EBV”).

As used herein, “culturing” skin fibroblasts is achieved, for example, by conducting the culturing at a temperature and in a growth factor milieu permissive of cell growth. In another embodiment, “culturing” skin fibroblasts is performed under conditions (e.g., those described below for proliferation) that preserve skin fibroblasts viability. In one embodiment, the temperature, humidity and protein milieu permissive of cell growth is 37° C., DMEM Medium with 10% fetal bovine serum (“FBS”) and 1% penicillin (“PS”). In one embodiment of this invention, the skin fibroblast-culturing step is performed for more than three hours. Preferably, the skin fibroblast-culturing step is performed for more than six hours (e.g., overnight).

Methods for obtaining skin fibroblasts from a subject's blood are known, and include, for example, skin punch biopsy, and growing cells out of explants. When cell confluence reaches 100%, cells are passaged. Typically after two passages, fibroblasts are purified in a proportion greater than 95%.

As used herein, cells “derived” from a subject are cells that arise through culturing and/or other physical manipulation performed on cells directly removed from the subject. For example, cultured skin fibroblasts derived from a subject are those skin fibroblasts that arise through culturing a sample of skin cells (e.g., contained in a punch biopsy) directly removed from the subject.

As used herein, “diagnosing Alzheimer's disease”, with respect to a symptomatic human subject, means determining that there is greater than 50% likelihood that the subject is afflicted with Alzheimer's disease. Preferably, “diagnosing Alzheimer's disease” means determining that there is greater than 60%, 70%, 80% or 90% likelihood that the subject is afflicted with Alzheimer's disease. As used herein, the phrase “determining whether the subject is afflicted with Alzheimer's disease” is synonymous with the phrase “diagnosing Alzheimer's disease.”

As used herein, “diagnosing non-ADD”, with respect to a symptomatic human subject, means determining that there is greater than 50% likelihood that the subject is afflicted with non-ADD. Preferably, “diagnosing non-ADD” means determining that there is greater than 60%, 70%, 80% or 90% likelihood that the subject is afflicted with non-ADD. As used herein, the phrase “determining whether the subject is afflicted with non-ADD” is synonymous with the phrase “diagnosing non-ADD.”

As used herein, “expression level”, with respect to a gene, includes, without limitation, any of the following: (i) the rate and/or degree of transcription of the gene (i.e., the rate at which, and/or degree to which, the gene is transcribed into RNA); (ii) the rate and/or degree of processing of the RNA encoded by the gene; (iii) the rate and/or degree of maturation of non-protein-coding RNA encoded by the gene; (iv) the rate at which, and/or degree to which, the RNA encoded by the gene is exported; (v) the rate at which, and/or degree to which, the RNA encoded by the gene is translated (i.e., the rate at which, and/or degree to which, the RNA is translated into protein); (vi) the rate at which, and/or degree to which, the protein encoded by the gene folds; (vii) the rate at which, and/or degree to which, the protein encoded by the gene is translocated; and (viii) the level of function (e.g., enzymatic activity or binding affinity) of the protein encoded by the gene.

As used herein, a gene is “differentially expressed between corresponding synchronized cells derived from AD patients and those derived from non-ADD patients” if, for example, the gene's TPM measure in synchronized cells derived from AD patients is different than in the same type of cells derived from non-ADD patients that are synchronized in the same way. For example, gene X would be differentially expressed between corresponding synchronized cells derived from AD patients and those derived from non-ADD patients if its TPM measure in synchronized cells derived from AD patients were 10 and its TPM measure were 100 in the same type of cells derived from non-ADD patients that are synchronized in the same way.

As used herein, an agent “favorably” affects the expression level of a gene whose expression level correlates with AD if it either decreases or increases that expression toward a level correlative with a non-AD (e.g., disease-free) state. For example, if the expression level of gene X is lower in an AD patient than in a non-afflicted patient, an agent favorably affecting the expression level of that gene would increase its expression level. Similarly, if the expression level of gene X is higher in an AD patient than in a non-afflicted patient, an agent favorably affecting the expression level of that gene would decrease its expression level.

As used herein, “measuring” the expression level of a gene means quantitatively determining the expression level via any means for doing so (e.g., Total RNA Sequencing (20 million reads, 2×75 bp PE)). Preferably, measuring the expression level of a gene is accomplished by measuring the number of RNA transcripts for that gene per million total RNA transcripts (i.e., “TPM” via FastQ data, and FPKM estimation per sample) present in the cell-derived RNA population being studied. For example, measuring the expression level of gene X in a synchronized cell population might yield a result of 50 TPM. In another embodiment, measuring a gene's expression level is done via protein quantification (e.g., via the known method of Western blotting). In a further embodiment, measuring a gene's expression level is done via a quantitative assay for protein function (e.g., via known methods for measuring enzymatic activity and/or protein binding strength).

As used herein, a subject afflicted with “non-Alzheimer's dementia” means a subject showing dementia such as, for example, that which characterizes Parkinson's disease, Huntington's disease and frontotemporal dementia.

As used herein, a “population” of cells includes any number of cells permitting the manipulation and study required to assess gene expression. In one embodiment, the population of cells includes at least 1,000,000 cells. In another embodiment, the population of cells includes between 100,000 cells and 1,000,000 cells, between 10,000 cells and 100,000 cells, between 1,000 cells and 10,000 cells, between 100 cells and 1,000 cells, between 10 cells and 100 cells, and fewer than 10 cells (e.g., one cell).

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 50 years or older, 55 years or older, 60 years or older, 65 or older, 70 or older, 75 or older, 80 or older, 85 or older, or 90 or older. The instant methods are envisioned for all subjects, preferably humans (and preferably symptomatic).

As used herein, a human subject who is “suspected of being afflicted with AD or non-ADD” is a subject displaying at least one symptom (e.g., dementia) consistent with both AD and non-ADD.

As used herein, “synchronizing” a population of cells means placing at least a majority of cells in that population in the same cell cycle stage (namely, in the G1, S, G2 or M stage, and preferably in the G1, S or G2 stage). In one embodiment, synchronizing a population of cells means placing at least 60%, at least 70%, at least 80%, at least 90%, at least 95% or preferably at least 99% of cells in that population in the same cell cycle stage. In another embodiment, synchronizing a population of cells means placing the cells in that population in the same cell cycle stage that they would be in if cultured to over-confluence and then starved. Cell confluence followed by serum starvation typically arrests the cells in the G0/G1 stage [1-3].

Doses, i.e., “therapeutically effective amounts”, used in connection with this invention include, for example, a single administration, and two or more administrations (i.e., fractions). In one embodiment, the therapeutically effective amount of a drug approved for a non-Alzheimer's indication is the dose and dosing regimen approved for that non-Alzheimer's indication.

As used herein, “treating” a subject afflicted with a disorder shall include, without limitation, (i) slowing, stopping or reversing the disorder's progression, (ii) slowing, stopping or reversing the progression of the disorder's symptoms, (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms.

The treatment of AD can be measured according to a number of clinical endpoints. These include, without limitation, (a) lowering, stabilizing or slowing progression of (i) dementia, (ii) synaptic loss, (iii) amyloid plaques and/or (iv) neurofibrillary tangles, and/or (b) favorably affecting the expression level of a gene whose expression level correlates with AD.

### Embodiments of the Invention

This invention provides accurate gene-based methods for determining whether a human subject is afflicted with AD or non-ADD when the subject is suspected of being afflicted with AD or non-ADD. The subject methods are based, at least in part, on the surprising discovery that synchronizing a patient's suitable cell population (e.g., lymphocytes, skin fibroblasts, pluripotent cells (such as iPSCs, and any progeny thereof)) and then measuring the expression levels of genes that are differentially expressed between AD and non-ADD cells permits accurately diagnosing the patient as having either AD or non-ADD. This invention also provides methods for treating AD using certain gene expression-altering agents.

Specifically, this invention provides a method for determining whether a human subject is afflicted with AD or non-ADD when the subject is suspected of being afflicted with AD or non-ADD, comprising the steps of
(a) synchronizing a population of suitable cells derived from the subject; and
(b) in the resulting synchronized cell population, measuring the expression level of a gene known to be differentially expressed between corresponding synchronized cells derived from AD patients and those derived from non-ADD patients,
   whereby (i) the subject is afflicted with AD if the expression level measured in step (b) is consistent with that gene's expression level in corresponding synchronized cells derived from AD patients, and (ii) the subject is afflicted with non-ADD if the expression level measured in step (b) is consistent with that gene's expression level in corresponding synchronized cells derived from non-ADD patients.

In one embodiment of the subject method, the suitable cells derived from the subject are cultured skin cell fibroblasts. In another embodiment, the suitable cells derived from the subject are cultured B lymphocytes (preferably immortalized B lymphocytes).

Methods for synchronizing cell populations are known in the art. In one embodiment of the subject method, synchronizing the population of suitable cells comprises culturing the cells to over-confluence and then starving the resulting over-confluent cells.

Ideally in the subject method, the gene is known to be differentially expressed by a significant margin. In one embodiment, the gene is known to be differentially expressed by at least 50% between corresponding synchronized cells derived from AD patients and those derived from non-ADD patients. Preferably, the gene is known to be differentially expressed by at least 100% between corresponding synchronized cells derived from AD patients and those derived from non-ADD patients. Another way of expressing the degree of differential expression is “% change” or “% Ch”, which is equal to [ADₑₓₚᵣₑₛₛᵢₒₙ−Non-ADDₑₓₚᵣₑₛₛᵢₒₙ/Non-ADDₑₓₚᵣₑₛₛᵢₒₙ].

In another preferred embodiment of the subject method, the gene is selected from the group consisting of CFAP97, LINC01393, ZNF623, HAUS2, PAN3, PSMB9, ZFP28, TTC26, RFESDP1, ZNF444, WASF2, NHLH1, NPPA-AS1_3, NORAD, URB2, ADAM20, ZCWPW2, AC004057.1, AC092651.1, ACP6, ACP2, C2CD5, CARNS1, FAM149B1, GLIS3-AS1, ASXL2 and IL18R1.

In one embodiment, the gene expression levels set forth in Table 9, taken individually or collectively (e.g., one, two or more, three or more, four or more, and the like), are indicative of AD. In a preferred embodiment, the gene expression levels set forth in Table 10, taken individually or collectively (e.g., one, two or more, three or more, four or more, and the like), are indicative of AD. For example, as shown in Table 10, a PSMB9 expression level greater than 18 TPM is indicative of AD. In yet another embodiment, AD-indicative expression levels for each other gene disclosed herein are readily determined based on the data presented.

In a further preferred embodiment of the subject method, step (b) comprises measuring the expression levels of a plurality of genes, each gene being known to be differentially expressed between corresponding synchronized cells derived from AD patients and those derived from non-ADD patients. The plurality of genes can be of any suitable size, such as at least two genes, at least five genes, at least 20 genes, at least 100 genes, and at least 1,000 genes. Preferably, each gene of the plurality of genes is known to be differentially expressed by at least 50% (and more preferably by at least 100%) between corresponding synchronized cells derived from AD patients and those derived from non-ADD patients. In yet another preferred embodiment of the subject method, the plurality of genes comprises two or more genes selected from the group consisting of AC004057.1, AC092651.1, ACP6, ADAM20, ASXL2, C2CD5, CARNS1, FAM149B1, GLIS3-AS1, IL18R1, LINC01393, LZIC, MAP1LC3B2, NHLH1, NORAD, NPPA-AS1_3, OSMR-AS1, PAN3, PHBP8, PSMB9, RAB3IP, RDH16, RFESDP1, RPL5, SCG2, SDHD, SHISA5, SLC45A3, SNHG14, TTC26, URB2, USMG5, WASF2, ZCWPW2, ZNF444, and ZNF70.

In the subject method where the expression levels of a plurality of genes are measured, the expression levels measured in step (b) are “consistent” with those in corresponding synchronized cells derived from AD patients if, for example, for at least a majority of gene expression levels measured, each such level is independently consistent with that gene's expression level in corresponding synchronized cells derived from AD patients.

In the subject method, measuring the expression level of a gene can be accomplished by any suitable method known in the art. In the preferred embodiment, measuring the expression level of a gene comprises measuring the number of that gene's RNA transcripts per number of total transcripts.

In a preferred embodiment, the subject invention provides a method for determining whether a human subject is afflicted with AD or non-ADD when the subject is suspected of being afflicted with AD or non-ADD, comprising the steps of
(a) synchronizing a population of cultured skin cell fibroblasts derived from the subject, wherein the synchronizing comprises culturing the fibroblasts to over-confluence and then starving the resulting over-confluent fibroblasts; and
(b) in the resulting synchronized fibroblast population, measuring the expression level of each of genes AC004057.1, AC092651.1, ACP6, ADAM20, ASXL2, C2CD5, CARNS1, FAM149B1, GLIS3-AS1, IL18R1, LINC01393, LZIC, MAP1LC3B2, NHLH1, NORAD, NPPA-AS1_3, OSMR-AS1, PAN3, PHBP8, PSMB9, RAB3IP, RDH16, RFESDP1, RPL5, SCG2, SDHD, SHISA5, SLC45A3, SNHG14, TTC26, URB2, USMG5, WASF2, ZCWPW2, ZNF444, and ZNF70, wherein measuring the expression level of each gene comprises measuring the number of its RNA transcripts per number of total transcripts.
   whereby (i) the subject is afflicted with AD if the expression levels measured in step (b) are consistent with the genes' expression levels in corresponding synchronized cells derived from AD patients, and (ii) the subject is afflicted with non-ADD if the expression levels measured in step (b) are consistent with the genes' expression levels in corresponding synchronized cells derived from non-ADD patients.

In another preferred embodiment, the subject invention provides a method for determining whether a human subject is afflicted with AD or non-ADD when the subject is suspected of being afflicted with AD or non-ADD, comprising the steps of
(a) synchronizing a population of cultured immortalized B lymphocytes derived from the subject, wherein the synchronizing comprises culturing the lymphocytes to over-confluence and then starving the resulting over-confluent lymphocytes; and
(b) in the resulting synchronized lymphocyte population, measuring the expression level of each of genes AC004057.1, AC092651.1, ACP6, ADAM20, ASXL2, C2CD5, CARNS1, FAM149B1, GLIS3-AS1, IL18R1, LINC01393, LZIC, MAP1LC3B2, NHLH1, NORAD, NPPA-AS1_3, OSMR-AS1, PAN3, PHBP8, PSMB9, RAB3IP, RDH16, RFESDP1, RPL5, SCG2, SDHD, SHISA5, SLC45A3, SNHG14, TTC26, URB2, USMG5, WASF2, ZCWPW2, ZNF444, and ZNF70 wherein measuring the expression level of each gene comprises measuring the number of its RNA transcripts per number of total transcripts.
   whereby (i) the subject is afflicted with AD if the expression levels measured in step (b) are consistent with the genes' expression levels in corresponding synchronized cells derived from AD patients, and (ii) the subject is afflicted with non-ADD if the expression levels measured in step (b) are consistent with the genes' expression levels in corresponding synchronized cells derived from non-ADD patients.

This invention further provides a method for determining whether a human subject is afflicted with AD, non-ADD, or a disorder which is neither (i.e., a non-demented subject (also referred to as “NDS”, “NDS patient”, “NDS subject”, “NDC” (i.e., non-demented control), “NDC patient”, and “NDC subject”)) when the subject is suspected of being afflicted with AD or non-ADD, comprising the steps of
(a) synchronizing a population of suitable cells derived from the subject; and
(b) in the resulting synchronized cell population, measuring the expression level of a gene known to be differentially expressed between corresponding synchronized cells derived from AD patients, those derived from non-ADD patients and those derived from NDS subjects.
   whereby (i) the subject is afflicted with AD if the expression level measured in step (b) is consistent with that gene's expression level in corresponding synchronized cells derived from AD patients, (ii) the subject is afflicted with non-ADD if the expression level measured in step (b) is consistent with that gene's expression level in corresponding synchronized cells derived from non-ADD patients, and (iii) the subject is afflicted with neither AD not non-ADD if the expression level measured in step (b) is consistent with that gene's expression level in corresponding synchronized cells derived from NDS subjects. The various embodiments of the diagnostic methods above for determining whether a human subject is afflicted with AD or non-ADD apply, mutatis mutandis, to this method.

This invention provides a method for determining whether a human subject is afflicted with AD or is a NDS when the subject is suspected of being afflicted with AD, comprising the steps of
(a) synchronizing a population of suitable cells derived from the subject; and
(b) in the resulting synchronized cell population, measuring the expression level of a gene known to be differentially expressed between corresponding synchronized cells derived from AD patients and those derived from NDS patients.
   whereby (i) the subject is afflicted with AD if the expression level measured in step (b) is consistent with that gene's expression level in corresponding synchronized cells derived from AD patients, and (ii) the subject is a NDS if the expression level measured in step (b) is consistent with that gene's expression level in corresponding synchronized cells derived from NDS patients.

This invention also provides a method for determining whether a human subject is afflicted with AD or is a NDS when the subject is not suspected of being afflicted with AD, comprising the steps of
(a) synchronizing a population of suitable cells derived from the subject; and
(b) in the resulting synchronized cell population, measuring the expression level of a gene known to be differentially expressed between corresponding synchronized cells derived from AD patients and those derived from NDS patients.
   whereby (i) the subject is afflicted with AD if the expression level measured in step (b) is consistent with that gene's expression level in corresponding synchronized cells derived from AD patients, and (ii) the subject is a NDS if the expression level measured in step (b) is consistent with that gene's expression level in corresponding synchronized cells derived from NDS patients.

In one embodiment of the subject method, the suitable cells derived from the subject are cultured skin cell fibroblasts. In another embodiment, the suitable cells derived from the subject are cultured B lymphocytes (preferably immortalized B lymphocytes).

Ideally in the subject method, the gene is known to be differentially expressed by a significant margin. In one embodiment, the gene is known to be differentially expressed by at least 50% between corresponding synchronized cells derived from AD patients and those derived from NDS patients. Preferably, the gene is known to be differentially expressed by at least 100% between corresponding synchronized cells derived from AD patients and those derived from NDS patients. Another way of expressing the degree of differential expression is “% change” or “% Ch”, which is equal to [ADₑₓₚᵣₑₛₛᵢₒₙ−NDSₑₓₚᵣₑₛₛᵢₒₙ/NDSₑₓₚᵣₑₛₛᵢₒₙ].

In a further preferred embodiment of the subject method, step (b) comprises measuring the expression levels of a plurality of genes, each gene being known to be differentially expressed between corresponding synchronized cells derived from AD patients and those derived from NDS patients. The plurality of genes can be of any suitable size, such as at least two genes, at least five genes, at least 20 genes, at least 100 genes, and at least 1,000 genes. Preferably, each gene of the plurality of genes is known to be differentially expressed by at least 50% (and more preferably by at least 100%) between corresponding synchronized cells derived from AD patients and those derived from NDS patients.

This invention further provides a method for treating a human subject afflicted with Alzheimer's disease comprising administering to the subject a therapeutically effective amount of an agent known to favorably affect the expression level of one or more genes whose expression levels correlate with Alzheimer's disease. Preferably, the genes are selected from the group consisting of AC004057.1, AC092651.1, ACP6, ADAM20, ASXL2, C2CD5, CARNS1, FAM149B1, GLIS3-AS1, IL18R1, LINC01393, LZIC, MAP1LC3B2, NHLH1, NORAD, NPPA-AS1_3, OSMR-AS1, PAN3, PHBP8, PSMB9, RAB3IP, RDH16, RFESDP1, RPL5, SCG2, SDHD, SHISA5, SLC45A3, SNHG14, TTC26, URB2, USMG5, WASF2, ZCWPW2, ZNF444, and ZNF70. In one embodiment, the genes are selected from the group consisting of IL18R1, PSMB9, TTC26, WASF2, ACP6, CARNS1, NPPA-AS1_3, SCG2 and SDHD. In another embodiment, the gene is IL18R1, PSMB9, TTC26, WASF2, ACP6, CARNS1, NPPA-AS1_3, SCG2 or SDHD.

This invention further provides methods for treating a human subject afflicted with Alzheimer's disease comprising administering to the subject a therapeutically effective amount of an agent selected from the group consisting of carfilzomib (Kyprolis®, Onyx Pharmaceuticals), bortezomib (Velcade®, Takeda Oncology), bumetanide (Bumex®, Hoffman-La Roche), furosemide (Lasix®, torsemide (Demadex®, flavin mononucleotide, phosphoric acid, riboflavin, gamma-aminobutyric acid, adenosine monophosphate, histidine, L-arginine, cisplatin, clozapine, cyclosporin A, dexamethasone, etanercept, ethanol, filgrastim, glucose, haloperidol, heparin, infliximab, leflunomide, nitric oxide, oxygen, polyethylene glycol, prednisolone, progesterone, tacrolimus, thalidomide, zinc, calcitriol, calcium, serine, acetylcholine, capsaicin, dopamine, histamine, lithium, norepinephrine, succinic acid, formic acid, tromethamine, citric acid, 10Z-hymenialdisine (Tocris), JIB 04 (Tocris), CRT 0066101 (Tocris), celastrol, dihydroeponemycin, noradrenaline bitartrate (Tocris), or any other drug listed in Tables 7A and 7B. In a preferred embodiment, the agent is carfilzomib which, in one embodiment, is administered in the manner stated on the FDA-approved label for one of its approved indications (e.g., in the manner approved for treating multiple myeloma, wherein the formulation is injectable and is administered at a dose of 30 mg or 60 mg). In another preferred embodiment, the agent is bortezomib which, in one embodiment, is administered in the manner stated on the FDA-approved label for one of its approved indications (e.g., in the manner approved for treating multiple myeloma, wherein the formulation is injectable and is administered at a dose of 3.5 mg, or 1.3 mg/m²). In another preferred embodiment, the agent is bumetanide which, in one embodiment, is administered in the manner stated on the FDA-approved label for one of its approved indications (e.g., in the manner approved for treating edema, wherein the formulation is oral and is administered at a dose of 0.5 mg, 1 mg or 2 mg daily, every other day, or daily for 3-4 days followed by a 1-2-day rest period). In another preferred embodiment, the agent is furosemide which, in one embodiment, is administered in the manner stated on the FDA-approved label for one of its approved indications (e.g., in the manner approved for treating edema or hypertension, wherein the formulation is oral and is administered at a dose of 20 mg, 40 mg, 60 mg or 80 mg per day (e.g., 40 mg 2×daily)). In another preferred embodiment, the agent is torsemide which, in one embodiment, is administered in the manner stated on the FDA-approved label for one of its approved indications (e.g., in the manner approved for treating edema or hypertension, wherein the formulation is oral and is administered at a dose of 5 mg, 10 mg, 15 mg or 20 mg per day).

In another preferred embodiment, the agent is any of cisplatin, clozapine, cyclosporin A, dexamethasone, etanercept, filgrastim, haloperidol, heparin, infliximab, leflunomide, prednisolone, progesterone, tacrolimus, thalidomide or calcitriol which, in one embodiment, is administered in the manner stated on the FDA-approved label for one of its approved indications.

As for each of 10Z-hymenialdisine, JIB 04, CRT 0066101, celastrol, dihydroeponemycin, noradrenaline bitartrate, and other non-FDA-approved drugs, the preferred route of administration is oral, and the preferred dosage is from 0.1 mg/kg to 100 mg/kg, from 1 mg/kg to 5 mg/kg, from 5 mg/kg to 10 mg/kg, from 10 mg/kg to 15 mg/kg, or from 15 mg/kg to 20 mg/kg.

This invention will be better understood by reference to the examples which follow, but those skilled in the art will readily appreciate that the specific examples detailed are only illustrative of the invention as described more fully in the claims which follow thereafter.

### EXAMPLES

### Example 1—Study 1

TABLE 1
Based on data from Study 1, the top 285 statistically significant genes with
less than 1% overlap probability between AD and Non-ADD.
Top 285 statistically significant genes with less than 1%
overlap probability between AD and Non-ADD

| | | | | |
|---|---|---|---|---|
| PTCD2 | UVSSA | SPIN2B | IFT80 | PLPP5 |
| ST20 | KIAA1551 | NPM1P50 | AL356277.2 | PCSK4 |
| AC090971.4 | SLC43A3 | MIR6501 | FUCA1 | PPP1R16B |
| EIF4A2P1 | BZW1P2 | MED26 | HIST1H2BL | PREPL |
| CFAP97 | HS6ST1P1 | ZNF860 | ZDHHC11B | ZNF106 |
| AL157871.3 | CYB561D2 | AC099508.1 | BATF2 | ZNF383 |
| AC007325.4 | SULT1A4 | MFN2 | RAET1E | LEAP2 |
| NR3C2 | AC138623.1 | ASAH1 | TUBB2B | ARHGAP42 |
| C6orf58 | RHPN1 | HDAC4 | FOXRED2 | NOXRED1 |
| LINC01393 | ST8SIA6 | ZNF628 | AK3P3 | SFXN5 |
| AL031432.5 | TTC8 | C16orf62 | WASF2 | AL365203.1 |
| AC005495.1 | IGDCC4 | AC025594.2 | LBHD1 | BX322639.1 |
| NR2C2 | XPC | ADAMTSL4-AS1 | BEX1 | AC005837.1 |
| AL589684.1 | TDO2 | TESK1 | CNOT6L | PVT1 |
| WDR17 | KBTBD6 | SFXN1 | ANKFY1 | AC073539.7 |
| FCF1P6 | AC004997.1 | JCHAIN | ACOX2 | PML |
| AC092818.1 | AL592183.1 | F2R | URAHP | KDELC2 |
| FGR | AC226101.1 | PLCB4 | COX7A2L | AC109583.2 |
| HNRNPA3P10 | AC087672.3 | CACUL1 | LSS | AL158835.2 |
| C17orf97 | FOXN3 | KRT8P33 | LINC02126 | AL591846.2 |
| FAM13A | KANK2 | IMPDH1P4 | AL049840.5 | DHRS4L2 |
| ARMCX5-GPRASP2 | ZNF107 | MIR4653 | PROCR | LINC02085 |
| ZNF274 | ZNF593 | AL590428.1 | AC097468.3 | TERF2 |
| IPO4 | AC093752.1 | ZFP28 | NHLH1 | PPP2R2D |
| CYB5D1 | CPVL | TTC26 | RAPGEF1 | PRORSD1P |
| AC005077.2 | TAS2R10 | TMIE | B3GALT5-AS1 | ABHD12 |
| ZNF547 | AC069528.2 | AP000766.1 | CCDC159 | AC010894.5 |
| HIC1 | SLC25A34 | ARPC5 | KLHL4 | DYNC1LI2 |
| LINC01239 | AC005674.2 | SIPA1 | MVD | AC015917.2 |
| MALRD1 | MAPK8IP3 | AP001830.1 | ELOVL6 | EIF3C |

| | | | | |
|---|---|---|---|---|
| HOXA-AS2 | ADD3 | TMEM167A | FAXC | FAM223A |
| NBR2 | VPS72 | RBSN | SYNPO | ZNF808 |
| AC010336.1 | CDC25B | CU634019.1 | AC087276.2 | PHF1 |
| KATNB1 | AC026464.3 | BTF3P9 | AC145343.1 | OR7E22P |
| BTBD7 | RASA3 | GSC | LRRC37A4P | AC027796.3 |
| UBE2FP1 | MIR6808 | CASTOR1 | Z97634.1 | AP001148.1 |
| NPPA-AS1_3 | SNORD36C | FLJ46284 | ZNF321P | LAMB2 |
| ANKRD36 | NMT1 | AC138150.2 | AP000763.2 | DMTN |
| AC124067.4 | EML2 | BEST4 | AC093270.1 | ASIC3 |
| DDIT4L | PRDM15 | AC002066.1 | AC021087.3 | PHBP19 |
| PTK2 | AC005363.1 | RPE | AL022328.4 | RNY1P16 |
| NORAD | MYNN | COLGALT2 | AKNA | AC022613.3 |
| COX6A1P2 | GSAP | NSMCE4A | CFAP43 | ZNF300 |
| UPK1A-AS1 | TNFSF12 | SNORD110 | SUMO2P6 | AF165147.1 |
| AC005521.1 | CATSPER2P1 | C1orf174 | HIST1H2BF | AC119403.1 |
| AC097532.2 | HSPA8P11 | PARP14 | SLIT1 | |
| EIF4BP5 | FOXK2 | AC007566.1 | ZNF407 | |
| HPCA | KIAA0556 | UBE2R2-AS1 | CAMK1 | |
| TCAF2P1 | ZNF688 | CACHD1 | MTCP1 | |
| URB2 | CU633904.1 | HCG20 | SKP1P1 | |
| MICB | SLC25A32 | AC091544.5 | ERI1 | |
| ORC1 | ATP6V1B2 | SNORD45A | PUM2 | |
| AP000238.1 | ZFP30 | AL031728.1 | CEP290 | |
| KDM7A | AL021707.8 | KIAA1468 | PLAC8 | |
| THAP7 | FNDC3A | JPT2 | SUPT16H | |
| FBXL17 | EIF3KP1 | AC095055.1 | HSF4 | |
| AL157895.1 | PNPLA7 | AC027097.1 | AC245052.3 | |
| TBC1D14 | GSTCD | AL356512.1 | PAN3 | |
| PPFIA4 | C8orf82 | FAM196B | KAT2A | |
| ANAPC13 | FZD1 | ARHGAP23 | FAM223B | |

(P < 0.01 - two-tailed, unequal variance T-test)

TABLE 2
Based on data from Study 1, genes with
functional relevance to PKC and MAPK

| Protein | Rank | Gene Name | T-test (2, 3) |
|---|---|---|---|
| Mitogen-Activated Protein | 60 | MAPK8IP3 | 0.0014 |
| Kinase 8 Interacting Protein 3 | | | |
| Heparin Binding EGF Like | 324 | HBEGF | 0.0112 |
| Growth Factor | | | |
| Heparan Sulfate Proteoglycan 2 | 369 | HSPG2 | 0.0138 |
| Solute Carrier Family 9 | 652 | SLC9A5 | 0.0278 |
| Member A5 | | | |
| Mitogen-Activated Protein | 747 | MAPK11 | 0.0323 |
| Kinase 11 | | | |
| Fos Proto-Oncogene, AP-1 | 892 | FOSL1P1 | 0.0383 |
| Transcription Factor Subunit | | | |

TABLE 3
Based on data from Study 1, genes with functional
relevance for cell adhesion and cell division

| Protein | Rank | Gene Name | T-test (2, 3) |
|---|---|---|---|
| Pentatricopeptide Repeat | 1 | PTCD2 | 0.0000 |
| Domain 2 | | | |
| Coiled-Coil Domain | 117 | CCDC159 | 0.0033 |
| Containing 159 | | | |
| Cell Division Cycle 25B | 183 | CDC25B | 0.0055 |
| TNF Superfamily Member 12 | 194 | TNFSF12 | 0.0061 |
| Glutathione S-Transferase | 208 | GSTCD | 0.0066 |
| C-Terminal Domain | | | |
| Containing | | | |
| TEN1-CDK3 Readthrough | 332 | TEN1-CDK3 | 0.0115 |
| (NMD Candidate) | | | |
| Programmed Cell Death 6 | 333 | PDCD6 | 0.0117 |
| CDC42 Effector Protein 5 | 407 | CDC42EP5 | 0.0157 |
| LMCD1 Antisense RNA 1 | 469 | LMCD1-AS1 | 0.0186 |
| (Head To Head) | | | |
| CD72 Molecule | 470 | CD72 | 0.0187 |
| Cell Division Cycle 37 | 531 | CDC37 | 0.0221 |
| Cyclin Dependent Kinase 2 | 550 | CDK2AP2P1 | 0.0232 |
| Associated Protein | | | |
| 2 Pseudogene 1 | | | |
| Coiled-Coil Domain | 553 | CCDC62 | 0.0234 |
| Containing 62 | | | |
| Coiled-Coil Domain | 649 | CCDC173 | 0.0277 |
| Containing 173 | | | |
| Interleukin 18 Receptor 1 | 703 | IL18R1 | 0.0298 |
| Adenomatosis Polyposis | 784 | APCDD1L | 0.0338 |
| Coli Down-Regulated | | | |
| 1-Like | | | |
| C2 Calcium Dependent | 840 | C2CD5 | 0.0363 |
| Domain Containing 5 | | | |
| Interleukin 17 Receptor D | 848 | IL17RD | 0.0366 |
| Coiled-Coil Domain | 909 | CCDC65 | 0.0393 |
| Containing 65 | | | |
| Cell Division Cycle 27 | 978 | CDC27P2 | 0.0432 |
| Pseudogene 2 | | | |
| Coiled-Coil Domain | 1006 | CCDC158 | 0.0446 |
| Containing 158 | | | |

### Example 2—Study 2; Synchronized Cell Cycle Gene Expression Test for Alzheimer's Disease; Cross-Validation of Genetic Differential Expression

The initial findings of the gene differential expression in synchronized skin fibroblasts, between the Alzheimer's Disease patients (AD; n=6) and the Non-Alzheimer's Disease Demented patients (Non-ADD; n=2), were cross-correlated with the second batch of samples (AD; n=2; Non-AD n=3). For the purpose of separating the two batches of samples, we called the first set of samples the “Training Set” and the second set of samples the “Validation Set.”

### Methods

The genes were ranked in decreasing statistical significance order, i.e., with the highest statistical significance first (examples in Tables 4 and 5). The ranking is based on the t-test (two tailed, unequal variance) for the two groups of samples AD and Non-ADD. The comparison of the two lists of genes was made as described below.

### Results

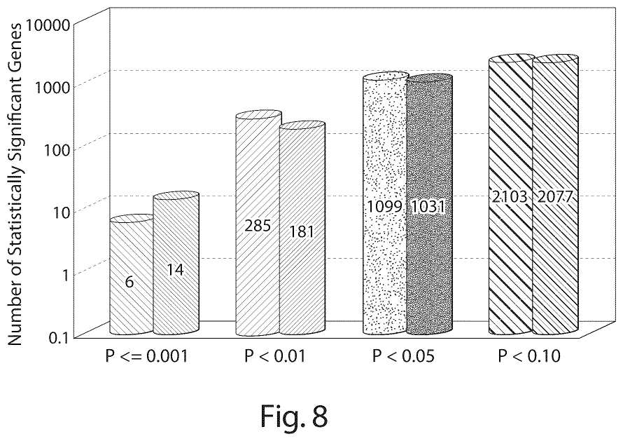

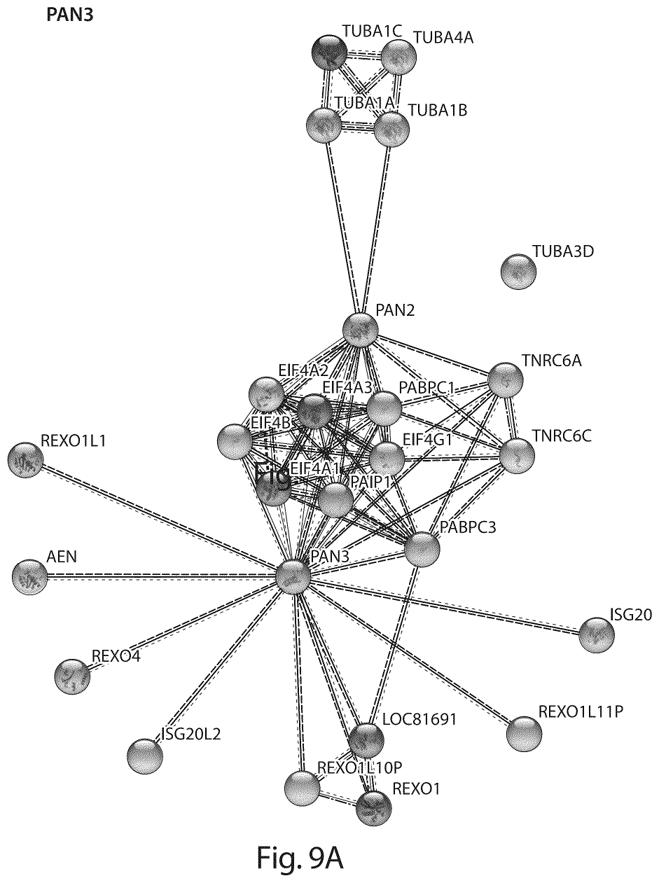

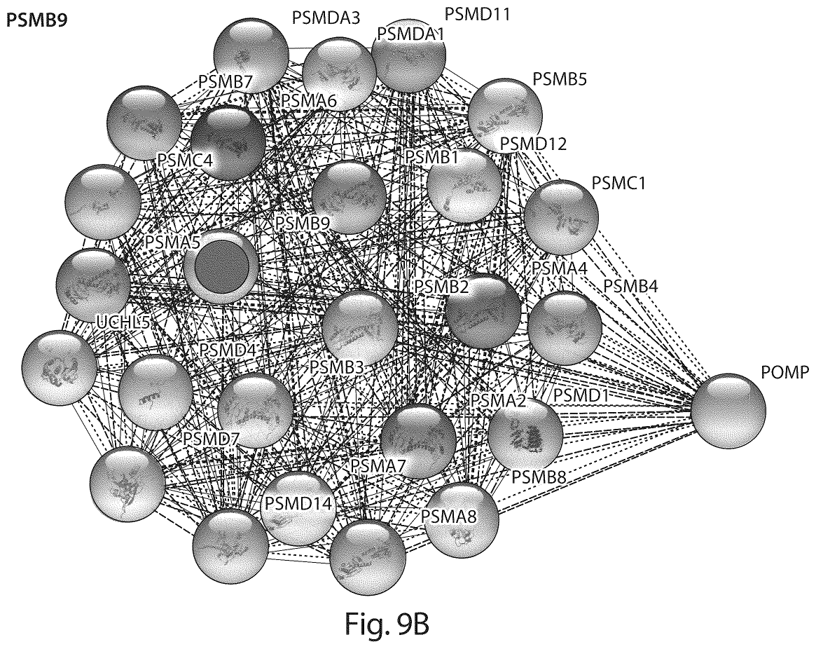

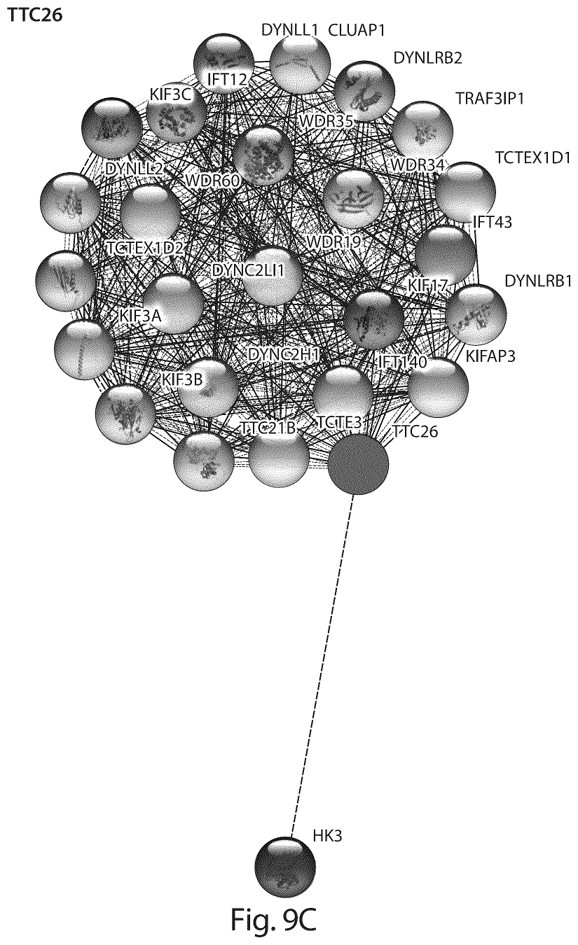

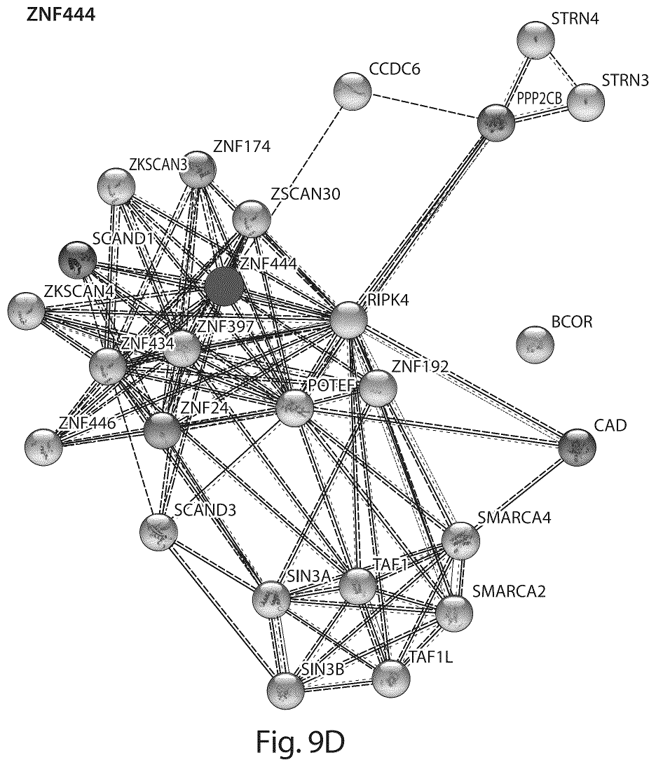

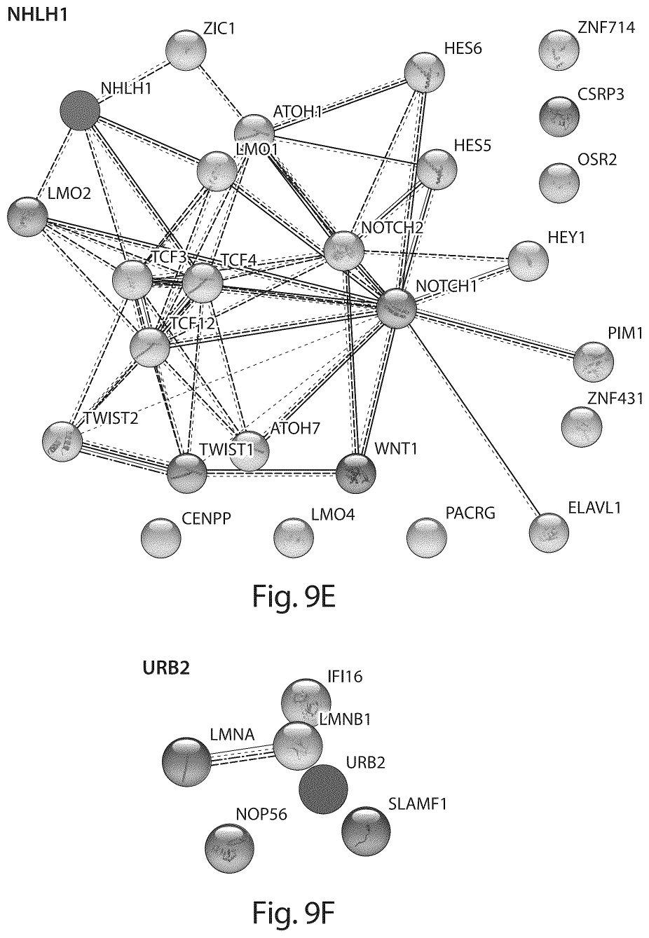

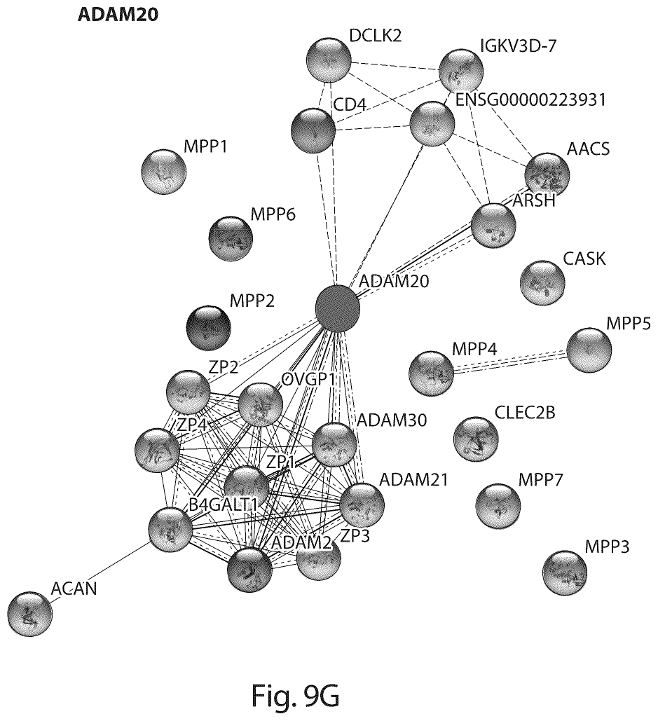

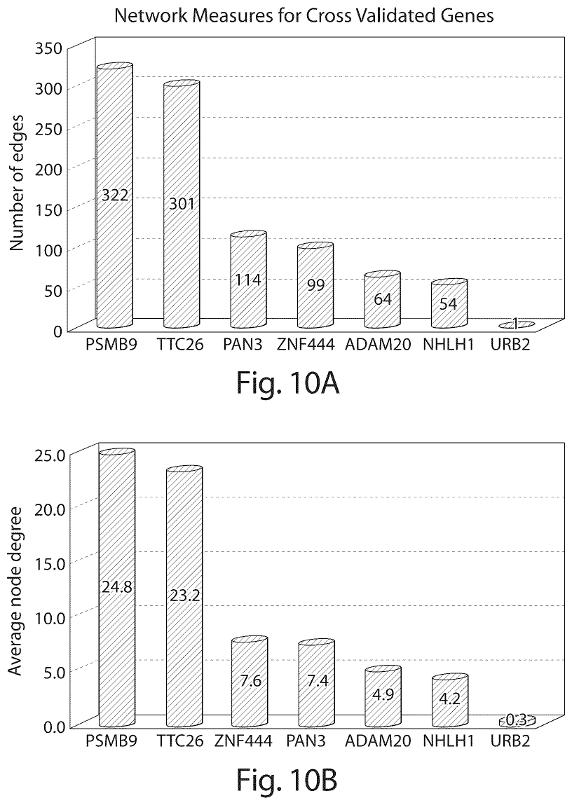

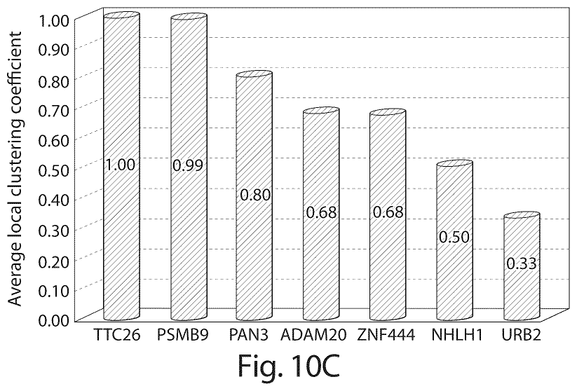

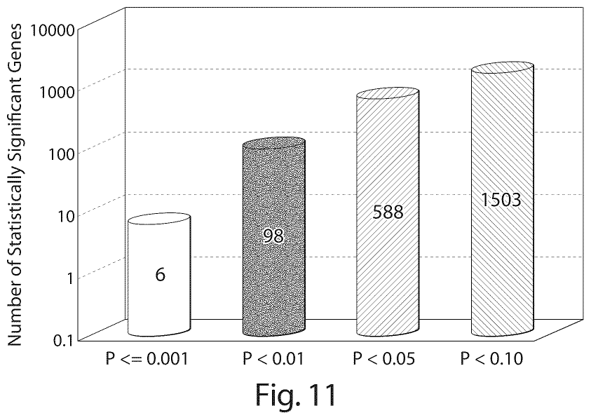

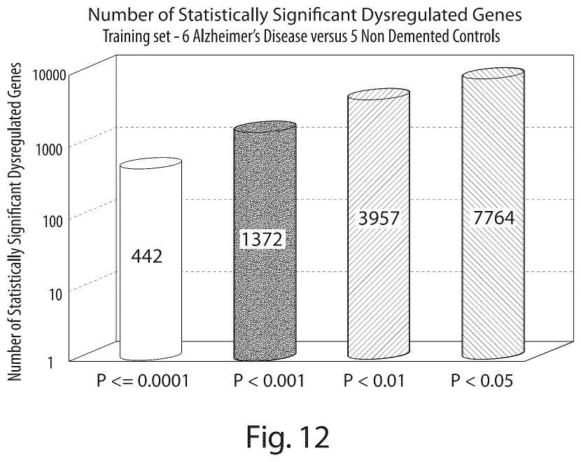

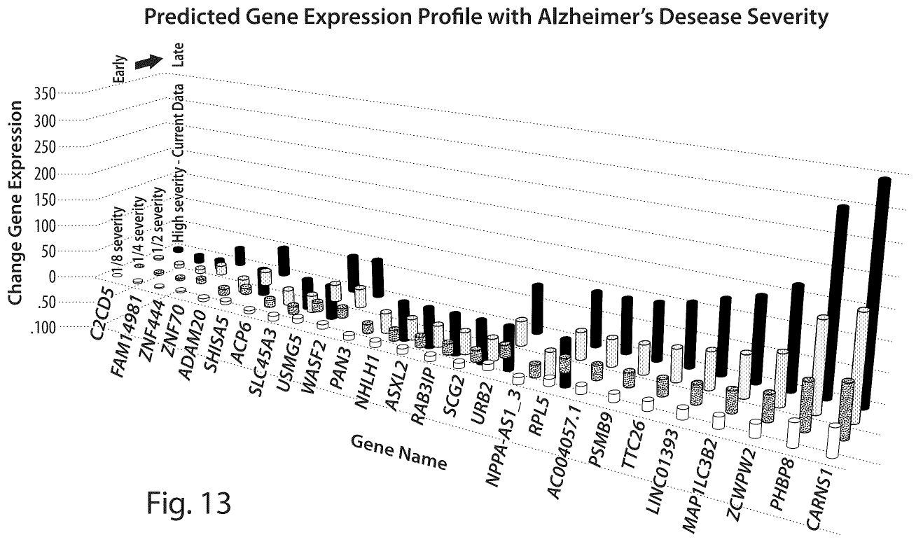

The number of statistically significant genes is similar in the training and validation sets (FIG. 8), with smaller differences for lower statistical significance (P<0.10) and larger differences for higher statistical significance (P<0.001). The larger difference for the higher statistical significance (P<0.001) could be due not only to the different number of samples in the validation set (5) when compared to the training set (8), but also to the different types of Non-ADD samples in the two sets. This difference suggests a high diversity of dysregulated pathways.

The majority of the genes (n=53) presented in Tables 4 and 5 are under highest statistical significance (P<0.001), and all of them are under high statistical significance (P<0.01). The presence of the first 40 genes from the training set (Table 4) was checked in the list of 2,077 genes from the validation set (P<0.10; FIG. 8). Similarly, the presence of the first 40 genes from the validation set (Table 5) was checked in the list of 2,103 genes from the training set (P<0.10; FIG. 8). The first 40 genes from Tables 4 and 5 are under highest statistical significance therefore it is very likely to have the highest impact in Alzheimer's disease detection, treatment, and pathways dysregulation. The cross-correlation of the first 40 genes in each set was made with a larger pool of genes from the opposite set (P<0.10) to accommodate the diversity in Non-ADD samples as well as to compensate for different numbers of samples in the validation (5) and training sets (8). However, in the end only the genes with similar statistical significance are considered as representing the core of dysregulation for AD.

The results of these initial findings in the highest statistically significant 40 genes suggests that about 81% of the genes which are dysregulated the training set are also dysregulated in the validation set. However, only about 7.5% of these genes show the same statistical significance in both training and validation set (Table 6).

Those genes showing the same statistical significance in the training and validation sets are at the core of the dysregulated pathways and will be very likely at the core of the genetic biomarkers for AD and at the core of the therapeutic targets for AD.

TABLE 4
Data for Differentially Expressed Genes from Study 2
First 40 differentially expressed genes in the
Training Set (6AD, 2 Non-ADD).
Training Set (6AD versus 2 Non-ADD)

| | | T-test Two tailed |
|---|---|---|
| Rank | Gene name | Unequal Variance |
| 1 | PTCD2 | 3.48E−05 |
| 2 | ST20 | 6.09E−05 |
| 3 | AC090971.4 | 9.92E−05 |
| 4 | EIF4A2P1 | 1.28E−04 |
| 5 | CFAP97 | 1.38E−04 |
| 6 | AL157871.3 | 1.44E−04 |
| 7 | AC007325.4 | 2.26E−04 |
| 8 | NR3C2 | 2.37E−04 |
| 9 | C6orf58 | 2.38E−04 |
| 10 | LINC01393 | 2.58E−04 |
| 11 | AL031432.5 | 2.87E−04 |
| 12 | AC005495.1 | 2.96E−04 |
| 13 | NR2C2 | 3.07E−04 |
| 14 | AL589684.1 | 3.26E−04 |
| 15 | WDR17 | 3.81E−04 |
| 16 | FCF1P6 | 4.04E−04 |
| 17 | AC092818.1 | 4.30E−04 |
| 18 | FGR | 4.36E−04 |
| 19 | HNRNPA3P10 | 4.41E−04 |
| 20 | C17orf97 | 4.73E−04 |
| 21 | FAM13A | 5.15E−04 |
| 22 | ARMCX5-GPRASP2 | 5.17E−04 |
| 23 | ZNF274 | 6.17E−04 |
| 24 | IPO4 | 6.62E−04 |
| 25 | CYB5D1 | 6.96E−04 |
| 26 | AC005077.2 | 7.29E−04 |
| 27 | ZNF547 | 7.57E−04 |
| 28 | HIC1 | 7.58E−04 |
| 29 | LINC01239 | 7.59E−04 |
| 30 | MALRD1 | 7.87E−04 |
| 31 | UVSSA | 8.09E−04 |
| 32 | KIAA1551 | 8.13E−04 |
| 33 | SLC43A3 | 8.17E−04 |
| 34 | BZW1P2 | 8.21E−04 |
| 35 | HS6ST1P1 | 8.89E−04 |
| 36 | CYB561D2 | 9.04E−04 |
| 37 | SULT1A4 | 9.57E−04 |
| 38 | AC138623.1 | 9.58E−04 |
| 39 | RHPN1 | 9.68E−04 |
| 40 | ST8SIA6 | 9.82E−04 |

TABLE 5
Data for Differentially Expressed Genes from Study 2
First 40 differentially expressed genes in the
Validation Set (2AD, 3 Non-ADD)
Validation Set (2 AD versus 3 Non-ADD)

| | | T-test Two tailed |
|---|---|---|
| Rank | Gene name | Unequal Variance |
| 1 | RPL13AP6 | 2.36E−05 |
| 2 | ARHGEF7 | 7.59E−05 |
| 3 | ZNF623 | 9.37E−05 |
| 4 | MYL12B | 3.47E−04 |
| 5 | RP11-500C11.3 | 3.79E−04 |
| 6 | EEF1A1P9 | 4.17E−04 |
| 7 | EIF3M | 5.39E−04 |
| 8 | NDUFB6 | 5.93E−04 |
| 9 | PGAM4 | 7.27E−04 |
| 10 | XXYLT1-AS2 | 8.29E−04 |
| 11 | PIGX | 8.60E−04 |
| 12 | FAM71F2 | 8.74E−04 |
| 13 | MPLKIP | 9.28E−04 |
| 14 | NDUFA8 | 9.30E−04 |
| 15 | TCP10L | 1.06E−03 |
| 16 | ATG9B | 1.09E−03 |
| 17 | FAM229B | 1.15E−03 |
| 18 | RPS18P12 | 1.15E−03 |
| 19 | RP3-340B19.2 | 1.16E−03 |
| 20 | SHFM1 | 1.27E−03 |
| 21 | bP-21264C1.2 | 1.31E−03 |
| 22 | FRMD5 | 1.37E−03 |
| 23 | ATOX1 | 1.39E−03 |
| 24 | ZCWPW1 | 1.43E−03 |
| 25 | NENF | 1.46E−03 |
| 26 | RPS15AP38 | 1.61E−03 |
| 27 | RP11-568N6.1 | 1.69E−03 |
| 28 | ZNF786 | 1.71E−03 |
| 29 | ZNF3 | 1.74E−03 |
| 30 | AP000688.14 | 1.75E−03 |
| 31 | RP5-1125A11.6 | 1.84E−03 |
| 32 | HAUS2 | 1.87E−03 |
| 33 | NDUFS1 | 1.95E−03 |
| 34 | CAPNS1 | 2.05E−03 |
| 35 | STEAP4 | 2.08E−03 |
| 36 | PAN3 | 2.09E−03 |
| 37 | RP5-940J5.6 | 2.10E−03 |
| 38 | RP11-266K4.14 | 2.10E−03 |
| 39 | ATP5L | 2.21E−03 |
| 40 | PSMB9 | 2.21E−03 |

TABLE 6
Data for Differentially Expressed Genes from Study 2
Differentially expressed genes with similar statistical significance
(P < 0.05; n = 36) in the Training and Validation sets.
Cross-Validated Genes under Statistical Significance of P < 0.05

| Number | Gene name | T-test Training | T-test Validation |
|---|---|---|---|
| 1 | AC004057.1 | 0.0246 | 0.0199 |
| 2 | AC092651.1 | 0.0307 | 0.0332 |
| 3 | ACP6 | 0.0332 | 0.0169 |
| 4 | ADAM20 | 0.0321 | 0.0082 |
| 5 | ASXL2 | 0.0397 | 0.0298 |
| 6 | C2CD5 | 0.0363 | 0.0256 |
| 7 | CARNS1 | 0.0281 | 0.0316 |
| 8 | FAM149B1 | 0.0370 | 0.0150 |
| 9 | GLIS3-AS1 | 0.0206 | 0.0409 |
| 10 | IL18R1 | 0.0298 | 0.0399 |
| 11 | LINC01393 | 0.0003 | 0.0115 |
| 12 | LZIC | 0.0338 | 0.0479 |
| 13 | MAP1LC3B2 | 0.0260 | 0.0271 |
| 14 | NHLH1 | 0.0032 | 0.0119 |
| 15 | NORAD | 0.0050 | 0.0424 |
| 16 | NPPA-AS1_3 | 0.0048 | 0.0080 |
| 17 | OSMR-AS1 | 0.0393 | 0.0241 |
| 18 | PAN3 | 0.0088 | 0.0021 |
| 19 | PHBP8 | 0.0256 | 0.0198 |
| 20 | PSMB9 | 0.0378 | 0.0022 |
| 21 | RAB3IP | 0.0137 | 0.0186 |
| 22 | RDH16 | 0.0117 | 0.0434 |
| 23 | RFESDP1 | 0.0237 | 0.0043 |
| 24 | RPL5 | 0.0220 | 0.0422 |
| 25 | SCG2 | 0.0408 | 0.0295 |
| 26 | SDHD | 0.0328 | 0.0463 |
| 27 | SHISA5 | 0.0188 | 0.0169 |
| 28 | SLC45A3 | 0.0265 | 0.0359 |
| 29 | SNHG14 | 0.0292 | 0.0259 |
| 30 | TTC26 | 0.0023 | 0.0269 |
| 31 | URB2 | 0.0051 | 0.0219 |
| 32 | USMG5 | 0.0384 | 0.0340 |
| 33 | WASF2 | 0.0027 | 0.0476 |
| 34 | ZCWPW2 | 0.0145 | 0.0107 |
| 35 | ZNF444 | 0.0158 | 0.0056 |
| 36 | ZNF70 | 0.0301 | 0.0311 |

TABLE 7A
Top Cross-Validated Genes (P < 0.05); Drugs, Disorders and Encoded Proteins (Study 2)
Top Cross-Validated Genes (P < 0.05); Drugs and Known Disorders and Phenotypes

| # | Gene name | Drugs | Company | Disorders and Phenotypes |
|---|---|---|---|---|
| 1 | AC004057.1 | NA | NA | Increased risk of |
| | alias for | | | cardiovascular disease |
| | RPS26P25 | | | (CVD) |
| 2 | AC092651.1 | NA | NA | Phenotype: bilirubin |
| | alias for | | | measurement, glomerular |
| | LOC100420889 | | | filtration rate, chronic |
| | | | | kidney disease |
| 3 | ACP6 | Flavin Mononucleotide | Pharma, | Schizophrenia, congenital |
| | | (Approved, | Nutra | heart disease (CHD) |
| | | Investigational), | | |
| | | Phosphoric acid | | |
| | | (Approved), Riboflavin | | |
| | | (Approved, | | |
| | | Investigational), 4- | | |
| | | Nitropheno (Experimental) | | |
| 4 | ADAM20 | NA | NA | May be involved in sperm |
| | | | | maturation and/or |
| | | | | fertilization; a disintegrin |
| | | | | and metalloprotease |
| | | | | (active) domain 20; |
| | | | | membrane anchored cell |
| | | | | surface adhesion protein; |
| | | | | testis-specific with similarity |
| | | | | to fertilin-alpha |
| 5 | ASXL2 | NA | NA | Shashi-Pena syndrome; |
| | | | | therapy-related |
| | | | | myelodysplastic syndrome; |
| | | | | ASXL2 and ASXL1 genes |
| | | | | were predicted cancer- |
| | | | | associated genes |
| 6 | C2CD5 | NA | NA | Dynamically associated |
| | | | | with GLUT4-containing |
| | | | | glucose storage vesicles |
| | | | | (GSV) and plasma |
| | | | | membrane in response to |
| | | | | insulin stimulation |
| 7 | CARNS1 | Gamma-Aminobutyric acid | Pharma, | Phenotype: mean |
| | | (Approved, | Nutra | corpuscular volume, mean |
| | | Investigational), | | corpuscular hemoglobin, |
| | | Phosphoric acid | | sunburn, body height, |
| | | (Approved), Adenosine | | histidine metabolism, |
| | | monophosphate | | homocarnosine |
| | | (Approved, | | biosynthesis, arginine and |
| | | Investigational), Histidine | | proline metabolism, beta- |
| | | (Approved), L-Arginin | | alanine metabolism |
| | | (Approved) | | (KEGG), lysine, |
| | | | | phenylalanine, tyrosine, |
| | | | | proline and tryptophan |
| | | | | catabolism |
| 8 | FAM149B1 | NA | NA | Phenotype: systolic blood |
| | | | | pressure, Heschl′s gyrus, a |
| | | | | core region of the auditory |
| | | | | cortex with highly variable |
| | | | | morphology, morphology |
| | | | | measurement |
| 9 | GLIS3-AS1 | NA | NA | Diabetes mellitus, |
| | | | | Neonatal, with congenital |
| | | | | hypothyroidism; ndh |
| | | | | syndrome neonatal |
| | | | | diabetes mellitus with |
| | | | | congenital hypothyroidism |
| | | | | neonatal diabetes- |
| | | | | congenital hypothyroidism- |
| | | | | congenital glaucoma- |
| | | | | hepatic fibrosis-polycystic |
| | | | | kidneys syndrome |
| 10 | IL18R1 | (43) Drugs for IL18R1 | Pharma, | Ordinary smallpox, Variola, |
| | | Gene, Cisplatin | Nutra | growth hormone |
| | | (Approved), Clozapine | | insensitivity syndrome, |
| | | (Approved), Cyclosporin A | | pituitary dwarfism, growth |
| | | (Approved, | | hormone receptor |
| | | Investigational), | | deficiency, laron dwarfism, |
| | | Dexamethasone | | laron-type isolated |
| | | (Approved, | | somatotropin defect, laron- |
| | | Investigational), | | type dwarfism, laron type |
| | | Etanercept (Approved, | | pituitary dwarfism, primary |
| | | Investigational), Ethanol | | growth hormone |
| | | (Approved), Filgrastim | | insensitivity, primary growth |
| | | (Approved), glucose | | hormone resistance, gh-r |
| | | (Approved), Haloperidol | | deficiency, growth hormone |
| | | (Approved), Heparin | | receptor defect, laron-type |
| | | (Approved, | | pituitary dwarfism, laron- |
| | | Investigational), Infliximab | | type short stature, primary |
| | | (Approved), Leflunomide | | gh resistance, severe gh |
| | | (Approved, | | insensitivity, complete |
| | | Investigational), Nitric | | growth hormone |
| | | Oxide (Approved), Oxygen | | insensitivity, gh receptor |
| | | (Approved), Polyethylene | | deficiency, primary gh |
| | | glycol (Approved), | | insensitivity, short stature |
| | | Prednisolone (Approved), | | due to growth hormone |
| | | Progesterone (Approved), | | resistance, lars, acute |
| | | Tacrolimus (Approved, | | basophilic leukemia, |
| | | Investigational), | | ehrlichiosis chafeensis, |
| | | Thalidomide (Approved, | | hme human ehrlichial |
| | | Investigational), Zinc | | infection, human monocytic |
| | | (Approved, | | type, pneumoconiosis, |
| | | Investigational), Calcitriol | | black lung, coal miner′s |
| | | (Approved), calcium | | pneumoconiosis, coal |
| | | (Approved), Serine | | workers′ lung, coal workers′ |
| | | (Approved), cyclic amp | | pneumoconiosis, |
| | | (Experimental), thymidine | | melanoedema, coal |
| | | (Experimental, | | worker′s pneumoconiosis, |
| | | Investigational), | | black lung disease, coal |
| | | Vesnarinone | | workers pneumoconiosis. |
| | | (Investigational), | | |
| | | Ceramide, estrogen, | | |
| | | LY294002, mometasone | | |
| | | furoate, NMDA, | | |
| | | Progestins, Rapamycin, | | |
| | | alanine, arginine, cysteine, | | |
| | | glutamine, leucine, | | |
| | | phenylalanine, proline, | | |
| | | threonine, tyrosine | | |
| 11 | LINC01393 | NA | NA | Phenotype: cytotoxicity |
| | | | | measurement, response to |
| | | | | clozapine, obesity |
| 12 | LZIC | NA | NA | Thiazolidinedione-induced |
| | | | | edema in diabetes. |
| | | | | Phenotype: leukocyte |
| | | | | count, systolic blood |
| | | | | pressure, resting heart rate |
| 13 | MAP1LC3B2 | NA | NA | Plays a role in mitophagy |
| | | | | which contributes to |
| | | | | regulating mitochondrial |
| | | | | quantity and quality by |
| | | | | eliminating the |
| | | | | mitochondria to a basal |
| | | | | level to fulfill cellular energy |
| | | | | requirements and |
| | | | | preventing excess ROS |
| | | | | production; whereas LC3s |
| | | | | are involved in elongation |
| | | | | of the phagophore |
| | | | | membrane, the GABARAP |
| 14 | NHLH1 | NA | NA | Cleft palate, isolated, |
| | | | | physical disorder, orofacial |
| | | | | cleft, cleft lip/palate- |
| | | | | ectodermal dysplasia |
| | | | | syndrome, split-hand/foot |
| | | | | malformation; May serve as |
| | | | | DNA-binding protein and |
| | | | | may be involved in the |
| | | | | control of cell-type |
| | | | | determination, possibly |
| | | | | within the developing |
| | | | | nervous system. Nascent |
| | | | | helix loop helix protein 1, |
| | | | | binding the E-box motif, |
| | | | | transiently expressed |
| | | | | during neurogenesis, |
| | | | | involved in retinal |
| | | | | development. Also |
| | | | | expressed in |
| | | | | neuroblastoma cell line |
| 15 | NORAD | Noradrenaline bitartrate | Tocris | Pancreatic cancer, bladder |
| | | | | cancer, esophageal cancer, |
| | | | | breast cancer, colorectal |
| | | | | cancer. Non-Coding RNA |
| | | | | activated by DNA damage |
| 16 | NPPA-AS1_3 | Bumetanide (Approved), | Genentech, | Atrial fibrillation, atrial |
| | | Furosemide (Approved), | Inc., Validus | standstill, atrial |
| | | Torsemide (Approved) | Pharmaceuticals | cardiomyopathy with heart |
| | | | LLC, Leo | block |
| | | | Pharma, Apotex | |
| | | | Corporation, | |
| | | | Sanis Health | |
| | | | Inc, Watson | |
| | | | Labs, Roche | |
| 17 | OSMR-AS1 | NA | NA | OSMR Antisense RNA 1 |
| 18 | PAN3 | 10Z-Hymenialdisine, JIB | Tocris | Phenotype: monocyte |
| | | 04, AZD 1208, G 5555, | | percentage of leukocytes, |
| | | CRT 0066101 | | granulocyte percentage of |
| | | | | myeloid white cells, |
| | | | | myeloid white cell count, |
| | | | | lymphocyte percentage of |
| | | | | leukocytes |
| 19 | PHBP8 | NA | NA | Adolescent idiopathic |
| | | | | scoliosis, total cholesterol |
| | | | | measurement, high density |
| | | | | lipoprotein cholesterol |
| | | | | measurement, Alzheimer′s |
| | | | | disease, hippocampal |
| | | | | volume |
| 20 | PSMB9 | Carfilzomib (Approved, | Amgen, | Proteasome-associated |
| | | Investigational), | Teva, Pfizer | autoinflammatory |
| | | Bortezomib (Approved, | | syndrome 3, eosinophilic |
| | | Investigational), Kyprolis | | variant of chromophobe |
| | | (Approved July 2012), | | renal cell carcinoma, |
| | | Celastrol, | | nasopharyngeal disease, |
| | | Dihydroeponemycin | | Waterhouse-Friderichsen |
| | | | | syndrome, cardiac |
| | | | | sarcoidosis, epstein-barr |
| | | | | virus-associated gastric |
| | | | | carcinoma |
| 21 | RAB3IP | NA | NA | Involved in actin |
| | | | | remodeling and polarized |
| | | | | membrane transport; |
| | | | | Diastolic blood pressure |
| | | | | and memory performance |
| 22 | RDH16 | Farnesol (Experimental), | NA | Platelet count, erythrocyte |
| | | NAD, Androstanediol, | | count, perceived |
| | | Androsterone | | unattractiveness to |
| | | | | mosquitos measurement |
| 23 | RFESDP1 | NA | NA | Chronic obstructive |
| | | | | pulmonary disease, |
| | | | | smoking cessation |
| 24 | RPL5 | Zinc (Approved, | NA | Mutations in this gene have |
| | | Investigational), | | been identified in patients |
| | | | | with Diamond-Blackfan |
| | | | | Anemia (DBA). |
| | | | | Hemangioma, interatrial |
| | | | | communication |
| 25 | SCG2 | Calcium (Approved), | Pharma, | Intracranial primitive |
| | | Acetylcholine (Approved), | Nutra | neuroectodermal tumor |
| | | Capsaicin (Approved), | | (intracranial |
| | | Dexamethasone | | pnet;intracranial primitive |
| | | (Approved, | | neuroectodermal |
| | | Investigational), Dopamine | | neoplasm), lymph node |
| | | (Approved), Glucose | | cancer (lymph node |
| | | (Approved), Histamine | | neoplasm, neoplasm of |
| | | (Approved, | | lymph node), collagenous |
| | | Investigational), Lithium | | colitis (microscopic colitis, |
| | | (Approved), | | collagenous type colitis, |
| | | Norepinephrine | | collagenous), |
| | | (Approved), Cyclic amp | | neuroendocrine tumor |
| | | (Experimental), ATP | | (neuroendocrine neoplasm, |
| | | (Investigational), 5- | | neuroendocrine carcinoma, |
| | | Hydroxytryptamine, | | neuroendocrine cancer, |
| | | Forskolin, Cysteine, | | neuroendocrine neoplasia, |
| | | Tyrosine | | carcinoma neuroendocrine, |
| | | | | neuroendocrine tumors, |
| | | | | carcinoma |
| | | | | neuroendocrine), |
| | | | | pheochromocytoma |
| | | | | (pheochromocytoma, |
| | | | | susceptibility to |
| | | | | pheochromocytoma, |
| | | | | modifier of |
| | | | | sporadic |
| | | | | pheochromocytoma/ |
| | | | | secreting paraganglioma |
| | | | | chromaffin cell tumor |
| | | | | medullary chromaffinoma |
| | | | | medullary paraganglioma |
| | | | | pheochromoblastoma |
| | | | | pcc chromaffin cell |
| | | | | neoplasm |
| | | | | pheochromocytoma, |
| | | | | malignant) |
| 26 | SDHD | Succinic acid (Approved), | Pharma, | Paraganglioma and gastric |
| | | Formic acid (Approved, | Nutra | stromal sarcoma, |
| | | Experimental, | | Paragangliomas, Cowden |
| | | Investigational), | | syndrome, mitochondrial |
| | | Tromethamine (Approved), | | complex ii deficiency, |
| | | Citric Acid (Approved) | | carcinoid tumors, intestinal; |
| | | | | hereditary paraganglioma- |
| | | | | pheochromocytoma |
| | | | | syndrome |
| 27 | SHISA5 | NA | NA | Vasculopathy, retinal, with |
| | | | | cerebral leukodystrophy, |
| | | | | aicardi-goutières syndrome |
| | | | | 1 (cree encephalitis, |
| | | | | aicardi-goutieres |
| | | | | syndrome, ags, |
| | | | | encephalopathy with basal |
| | | | | ganglia calcification, |
| | | | | encephalopathy with |
| | | | | intracranial calcification and |
| | | | | chronic lymphocytosis of |
| | | | | cerebrospinal fluid, |
| | | | | encephalopathy, familial |
| | | | | infantile, with calcification |
| | | | | of basal ganglia and |
| | | | | chronic cerebrospinal fluid |
| | | | | lymphocytosis |
| | | | | pseudotoxoplasmosis |
| | | | | syndrome familial infantile |
| | | | | encephalopathy with |
| | | | | intracranial calcification and |
| | | | | chronic cerebrospinal fluid |
| | | | | lymphocytosis) |
| 28 | SLC45A3 | NA | NA | Prostate cancer, |
| | | | | suppression of |
| | | | | tumorigenicity 12 (st12; |
| | | | | prostate adenocarcinoma |
| | | | | 1; pac1), male reproductive |
| | | | | organ cancer |
| 29 | SNHG14 | NA | NA | Angelman syndrome |
| | | | | (happy puppet syndrome), |
| | | | | Prader-Willi syndrome |
| | | | | (Prader-Labhart-Willi |
| | | | | syndrome), Gastric cancer |
| 30 | TTC26 | NA | NA | Joubert syndrome (Joubert- |
| | | | | boltshauser syndrome); |
| | | | | Cerebelloparenchymal |
| | | | | disorder, cerebellar vermis |
| | | | | agenesis, agenesis of |
| | | | | cerebellar vermis, |
| | | | | cerebello-oculo-renal |
| | | | | syndrome, familial aplasia |
| | | | | of the vermis, cerebello- |
| | | | | oculo-renal syndrome |
| 31 | URB2 | NA | NA | Hepatocellular carcinoma, |
| | | | | Buruli ulcer (buruli ulcer, |
| | | | | susceptibility to |
| | | | | mycobacterium ulcerans, |
| | | | | Bairnsdale ulcer, Daintree |
| | | | | ulcer, Mossman ulcer, |
| | | | | Searl ulcer). Phenotype: |
| | | | | red blood cell distribution |
| | | | | width, triglyceride |
| | | | | measurement, lipoprotein |
| | | | | cholesterol measurement, |
| | | | | high density lipoprotein |
| | | | | cholesterol measurement, |
| | | | | mean corpuscular |
| | | | | hemoglobin |
| 32 | USMG5 | NA | NA | Schizophrenia, autism |
| | alias for | | | spectrum disorder, worry |
| | ATP5MD | | | measurement, systemic |
| | | | | lupus erythematosus, |
| | | | | unipolar depression, |
| | | | | response to escitalopram, |
| | | | | response to citalopram, |
| | | | | mood disorder |
| 33 | WASF2 | Tyrosine | NA | Wiskott-Aldrich syndrome |
| | | | | (eczema- |
| | | | | thrombocytopenia- |
| | | | | immunodeficiency |
| | | | | syndrome), narcissistic |
| | | | | personality disorder, |
| | | | | substance abuse, tobacco |
| | | | | addiction, avoidant |
| | | | | personality disorder |
| | | | | (anxious personality |
| | | | | disorder) |
| 34 | ZCWPW2 | NA | NA | Multiple sclerosis, systolic |
| | | | | blood pressure, alcohol |
| | | | | drinking, uterine fibroid, |
| | | | | cognitive decline |
| 35 | ZNF444 | NA | NA | Chondrosarcoma, |
| | | | | extraskeletal myxoid |
| | | | | (extraskeletal myxoid |
| | | | | chondrosarcoma, myxoid |
| | | | | extraosseous |
| | | | | chondrosarcoma), coronary |
| | | | | artery disease, |
| | | | | microalbuminuria, |
| | | | | periodontitis, venous |
| | | | | thromboembolism |
| 36 | ZNF70 | NA | NA | Phenotype: serum IgG |
| | | | | glycosylation |
| | | | | measurement, fractional |
| | | | | shortening, parathyroid |
| | | | | hormone measurement, |
| | | | | ejection fraction |
| | | | | measurement, left |
| | | | | ventricular systolic function |
| | | | | measurement |

TABLE 7B
Top Cross-Validated Genes (P < 0.05); Drugs, Disorders and Encoded Proteins (Study 2)

| | Top Cross-Validated Genes (P < 0.05); Encoded Proteins | |
|---|---|---|
| # | Gene name | |
| 1 | AC004057.1 | Ankyrin 2, ANK2-212, 206, 205, 208, 202, 203, 202, 201, 214, 224, 227 |
| | alias for | |
| | RPS26P25 | |
| 2 | AC092651.1 | Anaphase Promoting Complex Subunit 1 Pseudogene. |
| | alias for | |
| | LOC100420889 | |
| 3 | ACP6 | This gene encodes a member of the histidine acid phosphatase protein |
| | | family. The encoded protein hydrolyzes lysophosphatidic acid, which is |
| | | involved in G protein-coupled receptor signaling, lipid raft modulation, |
| | | and in balancing lipid composition within the cell. Alternative splicing |
| | | results in multiple transcript variants. ACP6-001-Acid phosphatase 6 |
| 4 | ADAM20 | This gene encodes a member of the ADAM (a disintegrin and |
| | | metalloprotease domain) family. Members of this family are membrane- |
| | | anchored proteins structurally related to snake venom disintegrins, and |
| | | have been implicated in a variety of biological processes involving cell- |
| | | cell and cell-matrix interactions, including fertilization, muscle |
| | | development, and neurogenesis. The expression of this gene is testis- |
| | | specific. Disintegrin and metalloproteinase domain-containing protein 20 |
| 5 | ASXL2 | This gene encodes a member of a family of epigenetic regulators that |
| | | bind various histone-modifying enzymes and are involved in the |
| | | assembly of transcription factors at specific genomic loci. Naturally |
| | | occurring mutations in this gene are associated with cancer in several |
| | | tissue types (breast, bladder, pancreas, ovary, prostate, and blood). This |
| | | gene plays an important role in neurodevelopment, cardiac function, |
| | | adipogenesis, and osteoclastogenesis. Putative Polycomb group protein |
| | | ASXL2 |
| 6 | C2CD5 | C2 domain-containing protein 5 |
| 7 | CARNS1 | CARNS1 (EC 6.3.2.11), a member of the ATP-grasp family of ATPases, |
| | | catalyzes the formation of carnosine (beta-alanyl-L-histidine) and |
| | | homocarnosine (gamma-aminobutyryl-L-histidine), which are found |
| | | mainly in skeletal muscle and the central nervous system, respectively |
| | | (Drozak et al., 2010). Carnosine synthase 1. Catalyzes the synthesis of |
| | | carnosine and homocarnosine. Carnosine is synthesized more efficiently |
| | | than homocarnosine. |
| 8 | FAM149B1 | Protein FAM149B1. Predicted intracellular proteins |
| 9 | GLIS3-AS1 | GLIS3 Antisense RNA 1. |
| 10 | IL18R1 | The protein encoded by this gene is a cytokine receptor that belongs to |
| | | the interleukin 1 receptor family. This receptor specifically binds |
| | | interleukin 18 (IL18), and is essential for IL18 mediated signal |
| | | transduction. IFN-alpha and IL12 are reported to induce the expression |
| | | of this receptor in NK and T cells. This gene along with four other |
| | | members of the interleukin 1 receptor family, including IL1R2, IL1R1, |
| | | ILRL2 (IL-1Rrp2), and IL1RL1 (T1/ST2), form a gene cluster on |
| | | chromosome 2q. Alternatively spliced transcript variants encoding |
| | | different isoforms have been found for this gene. Interleukin-18 receptor 1. |
| 11 | LINC01393 | Long Intergenic Non-Protein Coding RNA 1393. |
| 12 | LZIC | Leucine zipper and CTNNBIP1 domain containing, isoform CRA_a. |
| | | Protein LZIC. |
| 13 | MAP1LC3B2 | Microtubule associated protein 1 light chain 3 beta 2. |
| 14 | NHLH1 | The helix-loop-helix (HLH) proteins are a family of putative transcription |
| | | factors, some of which have been shown to play an important role in |
| | | growth and development of a wide variety of tissues and species. Four |
| | | members of this family have been clearly implicated in tumorigenesis via |
| | | their involvement in chromosomal translocations in lymphoid tumors: |
| | | MYC (MIM 190080), LYL1 (MIM 151440), E2A (MIM 147141), and SCL |
| | | (MIM 187040). Helix-loop-helix protein 1. |
| 15 | NORAD | Non-Coding RNA Activated By DNA Damage. Lee et al. (2016) found |
| | | that DNA damage induced NORAD expression in a p53 (TP53; 191170)- |
| | | dependent manner in HCT116 human colon cancer cells. Conditional |
| | | knockout or knockdown of NORAD expression caused multiple mitotic |
| | | errors, including anaphase bridges, mitotic slippage, and significant |
| | | aneuploidy. Mass spectrometric analysis of proteins that bound to |
| | | NORAD identified Pumilio-2 (PUM2; 607205), a protein that destabilizes |
| | | mRNAs by binding to PREs in their 3-prime UTRs. NORAD functions as |
| | | a molecular decoy for Pumilio proteins and stabilizes Pumilio target |
| | | mRNAs. Using combined RNA antisense purification and quantitative |
| | | mass spectrometry, Munschauer et al. (2018) showed that NORAD |
| | | interacts with proteins involved in DNA replication and repair in steady- |
| | | state cells and localizes to the nucleus upon stimulation with replication |
| | | stress or DNA damage. Cells depleted for NORAD or RBMX displayed |
| | | an increased frequency of chromosome segregation defects, reduced |
| | | replication fork velocity, and altered cell-cycle progression. |
| 16 | NPPA-AS1_3 | Non-protein coding gene |
| 17 | OSMR-AS1 | An RNA Gene, and is affiliated with the non-coding RNA class. OSMR = |
| | | Oncostatin M receptor This gene encodes a member of the type I |
| | | cytokine receptor family. The encoded protein heterodimerizes with |
| | | interleukin 6 signal transducer to form the type II oncostatin M receptor |
| | | and with interleukin 31 receptor A to form the interleukin 31 receptor, |
| | | and thus transduces oncostatin M and interleukin 31 induced signaling |
| | | events. Mutations in this gene have been associated with familial |
| | | primary localized cutaneous amyloidosis. |
| 18 | PAN3 | PAN3 poly(A) specific ribonuclease subunit.PAB-dependent poly(A)- |
| | | specific ribonuclease subunit PAN3. The N-terminal zinc finger binds to |
| | | poly(A) RNA. Belongs to the protein kinase superfamily. PAN3 family. |
| | | Regulatory subunit of the poly(A)-nuclease (PAN) deadenylation |
| | | complex, one of two cytoplasmic mRNA deadenylases involved in |
| | | general and miRNA-mediated mRNA turnover. PAN specifically shortens |
| | | poly(A) tails of RNA and the activity is stimulated by poly(A)-binding |
| | | protein (PABP). PAN deadenylation is followed by rapid degradation of |
| | | the shortened mRNA tails by the CCR4-NOT complex. Deadenylated |
| | | mRNAs are then degraded by two alternative mechanisms, namely |
| | | exosome-mediated 3′-5′ exonucleolytic degradation, or deadenlyation- |
| | | dependent mRNA decapping and subsequent 5′-3′ exonucleolytic |
| | | degradation by XRN1. PAN3 acts as a positive regulator for PAN |
| | | activity, recruiting the catalytic subunit PAN2 to mRNA via its interaction |
| | | with RNA and PABP, and to miRNA targets via its interaction with |
| | | GW182 family proteins. |
| 19 | PHBP8 | Prohibitin Pseudogene 8. |
| 20 | PSMB9 | Proteasome subunit beta 9 |
| | | The proteasome is a multicatalytic proteinase complex with a highly |
| | | ordered ring-shaped 20S core structure. The core structure is composed |
| | | of 4 rings of 28 non-identical subunits; 2 rings are composed of 7 alpha |
| | | subunits and 2 rings are composed of 7 beta subunits. Proteasomes are |
| | | distributed throughout eukaryotic cells at a high concentration and |
| | | cleave peptides in an ATP/ubiquitin-dependent process in a non- |
| | | lysosomal pathway. An essential function of a modified proteasome, the |
| | | immunoproteasome, is the processing of class I MHC peptides. This |
| | | gene encodes a member of the proteasome B-type family, also known |
| | | as the T1B family, that is a 20S core beta subunit. This gene is located |
| | | in the class Il region of the MHC (major histocompatibility complex). |
| | | Expression of this gene is induced by gamma interferon and this gene |
| | | product replaces catalytic subunit 1 (proteasome beta 6 subunit) in the |
| | | immunoproteasome. Proteolytic processing is required to generate a |
| | | mature subunit. |
| 21 | RAB3IP | RAB3A interacting protein is a Protein Coding gene. Among its related |
| | | pathways are Cargo trafficking to the periciliary membrane and Vesicle- |
| | | mediated transport. |
| 22 | RDH16 | Retinol dehydrogenase 16 |
| 23 | RFESDP1 | Rieske (Fe—S) Domain Containing Pseudogene 1 is a Rieske (Fe—S) |
| | | Domain Containing Pseudogene 1. |
| 24 | RPL5 | Ribosomal protein L5. Ribosomes, the organelles that catalyze protein |
| | | synthesis, consist of a small 40S subunit and a large 60S subunit. |
| | | Together these subunits are composed of four RNA species and |
| | | approximately 80 structurally distinct proteins. This gene encodes a |
| | | member of the L18P family of ribosomal proteins and component of the |
| | | 60S subunit. The encoded protein binds 5S rRNA to form a stable |
| | | complex called the 5S ribonucleoprotein particle (RNP), which is |
| | | necessary for the transport of nonribosome-associated cytoplasmic 5S |
| | | RNA to the nucleolus for assembly into ribosomes. The encoded protein |
| | | may also function to inhibit tumorigenesis through the activation of |
| | | downstream tumor suppressors and the downregulation of oncoprotein |
| | | expression. Mutations in this gene have been identified in patients with |
| | | Diamond-Blackfan Anemia (DBA). This gene is co-transcribed with the |
| | | small nucleolar RNA gene U21, which is located in its fifth intron. As is |
| | | typical for genes encoding ribosomal proteins, there are multiple |
| | | processed pseudogenes of this gene dispersed throughout the genome. |
| 25 | SCG2 | Secretogranin II. The protein encoded by this gene is a member of the |
| | | chromogranin/secretogranin family of neuroendocrine secretory proteins. |
| | | Studies in rodents suggest that the full-length protein, secretogranin II, is |
| | | involved in the packaging or sorting of peptide hormones and |
| | | neuropeptides into secretory vesicles. The full-length protein is cleaved |
| | | to produce the active peptide secretoneurin, which exerts chemotaxic |
| | | effects on specific cell types, and EM66, whose function is unknown |
| 26 | SDHD | Succinate Dehydrogenase Complex Subunit D. This gene encodes a |
| | | member of complex II of the respiratory chain, which is responsible for |
| | | the oxidation of succinate. The encoded protein is one of two integral |
| | | membrane proteins anchoring the complex to the matrix side of the |
| | | mitochondrial inner membrane. Mutations in this gene are associated |
| | | with the formation of tumors, including hereditary paraganglioma. |
| | | Transmission of disease occurs almost exclusively through the paternal |
| | | allele, suggesting that this locus may be maternally imprinted. There are |
| | | pseudogenes for this gene on chromosomes 1, 2, 3, 7, and 18. |
| | | Alternative splicing results in multiple transcript variant |
| 27 | SHISA5 | Shisa family member 5. This gene encodes a member of the shisa |
| | | family. The encoded protein is localized to the endoplasmic reticulum, |
| | | and together with p53 induces apoptosis in a caspase-dependent |
| | | manner. Alternative splicing results in multiple transcript variants. |
| | | Related pseudogenes of this gene are found on chromosome X. Can |
| | | induce apoptosis in a caspase-dependent manner and plays a role in |
| | | p53/TP53-dependent apoptosis. Induced in a p53/TP53-dependent |
| | | manner in response to cellular stress. |
| 28 | SLC45A3 | Solute carrier family 45 member 3. Hexose transport. Transport of |
| | | glucose and other sugars, bile salts and organic acids, metal ions and |
| | | amine compounds. Phenotype: microRNAs in cancer, transcriptional |
| | | misregulation in cancer, and metabolism. |
| 29 | SNHG14 | Small Nucleolar RNA Host Gene 14. This gene is located within the |
| | | Prader-Willi Syndrome critical region on chromosome 15 and is |
| | | imprinted and expressed from the paternal allele. It encodes a |
| | | component of the small nuclear ribonucleoprotein complex, which |
| | | functions in pre-mRNA processing and may contribute to tissue-specific |
| | | alternative splicing. Alternative promoter use and alternative splicing |
| | | result in a multitude of transcript variants encoding the same protein. |
| | | Transcript variants that initiate at the CpG island-associated imprinting |
| | | center may be bicistronic and also encode the SNRPN upstream reading |
| | | frame protein (SNURF) from an upstream open reading frame. In |
| | | addition, long spliced transcripts for small nucleolar RNA host gene 14 |
| | | (SNHG14) may originate from the promoters at this locus and share |
| | | exons with this gene. Alterations in this region are associated with |
| | | parental imprint switch failure, which may cause Angelman syndrome or |
| | | Prader-Willi syndrome. |
| 30 | TTC26 | Tetratricopeptide Repeat Domain 26. Cytoplasmic expression in few |
| | | tissues, distinct in cilia. Component of the intraflagellar transport (IFT) |
| | | complex B required for transport of proteins in the motile cilium. |
| | | Required for transport of specific ciliary cargo proteins related to motility, |
| | | while it is neither required for IFT complex B assembly or motion nor for |
| | | cilium assembly. Required for efficient coupling between the |
| | | accumulation of GLI2 and GLI3 at the ciliary tips and their dissociation |
| | | from the negative regulator SUFU. Plays a key role in maintaining the |
| | | integrity of the IFT complex B and the proper ciliary localization of the |
| | | IFT complex B components. Not required for IFT complex A ciliary |
| | | localization or function. Essential for maintaining proper microtubule |
| | | organization within the ciliary axoneme. |
| 31 | URB2 | URB2 ribosome biogenesis 2 homolog. High density |
| 32 | USMG5 alias | Up-regulated during skeletal muscle growth 5 homolog. Plays a critical |
| | for ATP5MD | role in maintaining the ATP synthase population in mitochondria. |
| | | USMG5_HUMAN, Q96IX5 Transactivated by SBP1. |
| 33 | WASF2 | WASP Family Member 2, This gene encodes a member of the Wiskott- |
| | | Aldrich syndrome protein family. The gene product is a protein that |
| | | forms a multiprotein complex that links receptor kinases and actin. |
| | | Binding to actin occurs through a C-terminal verprolin homology domain |
| | | in all family members. The multiprotein complex serves to tranduce |
| | | signals that involve changes in cell shape, motility or function. The |
| | | published map location (PMID: 10381382) has been changed based on |
| | | recent genomic sequence comparisons, which indicate that the |
| | | expressed gene is located on chromosome 1, and a pseudogene may |
| | | be located on chromosome X. Two transcript variants encoding different |
| | | isoforms have been found for this gene. Downstream effector molecule |
| | | involved in the transmission of signals from tyrosine kinase receptors |
| | | and small GTPases to the actin cytoskeleton. Promotes formation of |
| | | actin filaments. Part of the WAVE complex that regulates lamellipodia |
| | | formation. The WAVE complex regulates actin filament reorganization |
| | | via its interaction with the Arp2/3 complex. |
| 34 | ZCWPW2 | Zinc Finger CW-Type And PWWP Domain Containing 2. |
| 35 | ZNF444 | Zinc finger protein 444. This gene encodes a zinc finger protein that |
| | | activates transcription of a scavenger receptor gene involved in the |
| | | degradation of acetylated low density lipoprotein (Ac-LDL) (PMID: |
| | | 11978792). This gene is located in a cluster of zinc finger genes on |
| | | chromosome 19 at q13.4. A pseudogene of this gene is located on |
| | | chromosome 15. Multiple transcript variants encoding different isoforms |
| | | have been found for this gene. |
| 36 | ZNF70 | Zinc Finger Protein 70. May be involved in transcriptional regulation. |

TABLE 8
Protein Networks (Study 2)

| | | | | | Average |
|---|---|---|---|---|---|
| | | | | Average | local |
| | | Number | Number | node | clustering |
| Number | Gene name | of nodes | of edges | degree | coefficient |
| 1 | AC004057.1 | NA | NA | NA | NA |
| 2 | AC092651.1 | NA | NA | NA | NA |
| 3 | ADAM20 | 26 | 64 | 4.92 | 0.681 |
| 4 | ASXL2 | 26 | 212 | 16.3 | 0.876 |
| 5 | C2CD5 | 26 | 239 | 18.4 | 0.98 |
| 6 | CARNS1 | 26 | 209 | 16.1 | 0.84 |
| 7 | FAM149B1 | 2 | 1 | 1 | 1 |
| 8 | GLIS3- | NA | NA | NA | NA |
| | AS1FAM149B1 | | | | |
| 9 | IL18R1 | 20 | 90 | 9 | 0.768 |
| 10 | LINC01393 | NA | NA | NA | NA |
| 11 | LINC01393 | NA | NA | NA | NA |
| 12 | LZIC | 2 | 1 | 1 | 1 |
| 13 | MAP1LC3B2 | NA | NA | NA | NA |
| 14 | NHLH1 | 26 | 54 | 4.15 | 0.504 |
| 15 | NORAD | NA | NA | NA | NA |
| 16 | NPPA-AS1_3 | NA | NA | NA | NA |
| 17 | OSMR-AS1 | NA | NA | NA | NA |
| 18 | PAN3 | 31 | 114 | 7.35 | 0.801 |
| 19 | PHBP8 | NA | NA | NA | NA |
| 20 | PSMB9 | 26 | 322 | 24.8 | 0.992 |
| 21 | RAB3IP | 26 | 301 | 23.2 | 0.997 |
| 22 | RDH16 | 26 | 39 | 3 | 0.613 |
| 23 | RFESDP1 | NA | NA | NA | NA |
| 24 | RPL5 | 26 | 323 | 24.8 | 0.994 |
| 25 | SCG2 | 18 | 27 | 3 | 0.586 |
| 26 | SDHD | 26 | 115 | 8.85 | 0.769 |
| 27 | SHISA5 | 10 | 5 | 1 | 0.6 |
| 28 | SLC45A3 | 14 | 6 | 0.857 | 0.381 |
| 29 | SNHG14 | NA | NA | NA | NA |
| 30 | TTC26 | 26 | 301 | 23.2 | 0.997 |
| 31 | URB2 | 6 | 1 | 0.333 | 0.333 |
| 32 | USMG5 | 26 | 325 | 25 | 1 |
| 33 | WASF2 | 51 | NA | NA | NA |
| 34 | ZCWPW2 | NA | NA | NA | NA |
| 35 | ZNF444 | 26 | 99 | 7.62 | 0.675 |
| 36 | ZNF70 | 26 | 126 | 9.69 | 0.612 |

### Example 3—Study 2; TPM Values

### Reference Intervals

The average and standard deviations were calculated for the transcripts per million (TPM) values for each of the two groups—Alzheimer's disease (AD) and Non-Alzheimer's Disease Demented (Non-ADD) for each gene. The reference intervals were then calculated according to Horn and Pesce (Reference Intervals: A User's Guide. Paul S. Horn and Amadeo J. Pesce. Washington, DC: AACC Press, 2005, ISBN 1-59425-035-9) as the average plus minus two standard deviations. The reference intervals calculated in this way assure that 95% of all the possible values in each population (AD or non-ADD) are considered.

### Gap Between AD and Non-ADD

If there is no overlap between the reference intervals of AD and Non-ADD, there is a gap between the two bell-shaped curves and that indicates unequivocal diagnosis.

If there is an overlap in the reference intervals for AD and Non-ADD (light grey in Table 9), then there is a possibility of having a false positive or a false negative in the diagnosis. The genes that show overlap in the reference intervals, i.e., no gap (light grey) were eliminated from the final vector diagnosis. The genes that show an average of zero in one of the groups, either in the AD group or in the Non-ADD group, were also eliminated.

### Cut-Off for Each Gene

The cut-offs for each of the remaining 26 genes (Table 10) was determined as the middle of the gap in the reference intervals.

### Genetic Vector AD Diagnosis

The AD diagnosis is based on the 26 components/genes of the vector. For each one of the components, the greater than (>) or smaller than (<) the cut-off value is indicated for each gene, in the last column.

TABLE 9
Gene Expression Levels (TPM) Indicative of AD
(Study 2)

| Number | Gene name | Cut-Off | AD Diagnosis |
|---|---|---|---|
| 1 | AC004057.1 | 161.81 | >161.81 |
| 2* | AC092651.1 | 5.40 | >5.40 |
| 3 | ACP6 | 2.84 | <2.84 |
| 4 | ADAM20 | 0.16 | <0.16 |
| 5 | ASXL2 | 0.74 | <0.76 |
| 6 | C2CD5 | 28.76 | >28.76 |
| 7 | CARNS1 | 0.16 | >0.15 |
| 8 | FAM149B1 | 22.60 | <22.60 |
| 9** | GLIS3-AS1 | 0.11 | >0.11 |
| 10* | IL18R1 | 1.21 | <0.88 |
| 11 | LINC01393 | 1.00 | >0.86 |
| 12* | LZIC | 7.14 | >7.14 |
| 13 | MAP1LC3B2 | 4.41 | >4.41 |
| 14 | NHLH1 | 0.27 | <0.27 |
| 15* | NORAD | 61.03 | >61.03 |
| 16 | NPPA-AS1_3 | 2.54 | <2.54 |
| 17* | OSMR-AS1 | 1.37 | >1.37 |
| 18 | PAN3 | 15.98 | <15.98 |
| 19 | PHBP8 | 0.98 | >0.98 |
| 20 | PSMB9 | 18.00 | >18.00 |
| 21 | RAB3IP | 0.50 | <0.50 |
| 22* | RDH16 | 0.58 | <0.58 |
| 23** | RFESDP1 | 0.14 | <0.00 |
| 24 | RPL5 | 794.93 | >794.88 |
| 25 | SCG2 | 0.68 | <0.68 |
| 26* | SDHD | 40.92 | >40.92 |
| 27 | SHISA5 | 107.32 | >107.32 |
| 28 | SLC45A3 | 1.11 | <1.11 |
| 29* | SNHG14 | 26.70 | <26.70 |
| 30 | TTC26 | 2.41 | >2.41 |
| 31 | URB2 | 2.28 | >2.28 |
| 32 | USMG5 | 129.13 | >129.13 |
| 33 | WASF2 | 23.74 | >23.74 |
| 34 | ZCWPW2 | 1.15 | >1.15 |
| 35 | ZNF444 | 17.60 | <16.73 |
| 36 | ZNF70 | 0.87 | >0.87 |

*no gap
**zero average in one group

TABLE 10A
Gene Expression Levels (TPM) Indicative of AD
(Study 2)

| Number | Gene name | Cut-Off | AD Diagnosis |
|---|---|---|---|
| 1 | AC004057.1 | 161.81 | >161.81 |
| 2 | ACP6 | 2.84 | <2.84 |
| 3 | ADAM20 | 0.16 | <0.16 |
| 4 | ASXL2 | 0.74 | <0.76 |
| 5 | C2CD5 | 28.76 | >28.76 |
| 6 | CARNS1 | 0.16 | >0.15 |
| 7 | FAM149B1 | 22.60 | <22.60 |
| 8 | LINC01393 | 1.00 | >0.86 |
| 9 | MAP1LC3B2 | 4.41 | >4.41 |
| 10 | NHLH1 | 0.27 | <0.27 |
| 11 | NPPA-AS1_3 | 2.54 | <2.54 |
| 12 | PAN3 | 15.98 | <15.98 |
| 13 | PHBP8 | 0.98 | >0.98 |
| 14 | PSMB9 | 18.00 | >18.00 |
| 15 | RAB3IP | 0.50 | <0.50 |
| 16 | RPL5 | 794.93 | >794.88 |
| 17 | SCG2 | 0.68 | <0.68 |
| 18 | SHISA5 | 107.32 | >107.32 |
| 19 | SLC45A3 | 1.11 | <1.11 |
| 20 | TTC26 | 2.41 | >2.41 |
| 21 | URB2 | 2.28 | >2.28 |
| 22 | USMG5 | 129.13 | >129.13 |
| 23 | WASF2 | 23.74 | >23.74 |
| 24 | ZCWPW2 | 1.15 | >1.15 |
| 25 | ZNF444 | 17.60 | <16.73 |
| 26 | ZNF70 | 0.87 | >0.87 |

TABLE 10B
Gene Expression Levels (TPM) Indicative of AD
(Study 2) (Ranked According to % Change)

| Rank | Gene Name | Cut-Off | AD Diag. | % Change |
|---|---|---|---|---|
| 1 | CARNS1 | 0.16 | >0.15 | 347 |
| 2 | PHBP8 | 0.98 | >0.98 | 299 |
| 3 | ZCWPW2 | 1.15 | >1.15 | 170 |
| 4 | MAP1LC3B2 | 4.41 | >4.41 | 142 |
| 5 | LINC01393 | 1 | >0.86 | 127 |
| 6 | TTC26 | 2.41 | >2.41 | 110 |
| 7 | PSMB9 | 18 | >18.00 | 99 |
| 8 | AC004057.1 | 161.81 | >161.81 | 94 |
| 9 | RPL5 | 794.93 | >794.88 | 94 |
| 10 | NPPA-AS1_3 | 2.54 | <2.54 | −86 |
| 11 | URB2 | 2.28 | >2.28 | 85 |
| 12 | SCG2 | 0.68 | <0.68 | −81 |
| 13 | RAB3IP | 0.5 | <0.50 | −80 |
| 14 | ASXL2 | 0.74 | <0.76 | −77 |
| 15 | NHLH1 | 0.27 | <0.27 | −75 |
| 16 | PAN3 | 15.98 | <15.98 | −73 |
| 17 | WASF2 | 23.74 | >23.74 | 69 |
| 18 | USMG5 | 129.13 | >129.13 | 66 |
| 19 | SLC45A3 | 1.11 | <1.11 | −65 |
| 20 | ACP6 | 2.84 | <2.84 | −57 |
| 21 | SHISA5 | 107.32 | >107.32 | 53 |
| 22 | ADAM20 | 0.16 | <0.16 | −51 |
| 23 | ZNF70 | 0.87 | >0.87 | 34 |
| 24 | ZNF444 | 17.6 | <16.73 | −18 |
| 25 | FAM149B1 | 22.6 | <22.60 | −16 |
| 26 | C2CD5 | 28.76 | >28.76 | 3 |

TABLE 11
AD/NDC Dysregulated Genes under a Statistical Significance of P < 0.05
AD vs NDC; Statistical Significance T-test, to tailed, unequal variance

| # | Gene name | Training Set | Validation Set |
|---|---|---|---|
| 1 | ACIN1 | 9.77E−05 | 2.39E−02 |
| 2 | ACO2 | 1.40E−02 | 3.54E−02 |
| 3 | ACSL4 | 4.02E−05 | 1.25E−03 |
| 4 | ACTR1A | 8.44E−03 | 3.92E−02 |
| 5 | ADAM20 | 3.17E−02 | 2.08E−02 |
| 6 | ADAMTS14 | 2.85E−02 | 3.31E−02 |
| 7 | ADIPOR2 | 5.62E−03 | 4.83E−02 |
| 8 | AHCY | 1.24E−04 | 1.40E−02 |
| 9 | AL591845.1 | 2.86E−02 | 4.82E−02 |
| 10 | ALG5 | 7.60E−04 | 4.24E−02 |
| 11 | AMMECR1 | 2.48E−02 | 1.72E−02 |
| 12 | ANAPC13 | 2.54E−04 | 4.01E−02 |
| 13 | ANGPTL1 | 4.34E−02 | 4.87E−02 |
| 14 | ANXA7 | 7.94E−03 | 4.77E−02 |
| 15 | AOX1 | 3.60E−02 | 1.93E−03 |
| 16 | ARAP3 | 1.09E−02 | 1.68E−02 |
| 17 | ARF3 | 4.13E−06 | 6.92E−03 |
| 18 | ARPP19 | 1.77E−04 | 4.06E−02 |
| 19 | ARSD | 6.72E−03 | 3.52E−02 |
| 20 | ASAP3 | 1.98E−02 | 1.65E−02 |
| 21 | ASTE1 | 2.71E−03 | 1.54E−02 |
| 22 | ATF6 | 1.02E−02 | 2.17E−02 |
| 23 | ATP11B | 3.43E−02 | 1.98E−02 |
| 24 | ATP5F1 | 2.43E−02 | 8.83E−03 |
| 25 | AURKC | 3.23E−04 | 2.34E−02 |
| 26 | AVPI1 | 1.14E−02 | 1.20E−02 |
| 27 | B4GALT4 | 2.79E−03 | 2.86E−02 |
| 28 | BCORL1 | 2.53E−02 | 3.19E−02 |
| 29 | BICC1 | 1.24E−03 | 7.26E−03 |
| 30 | BLVRA | 7.50E−05 | 2.98E−02 |
| 31 | BTBD1 | 3.78E−03 | 1.29E−02 |
| 32 | BZW2 | 1.19E−03 | 4.23E−03 |
| 33 | C11orf63 | 1.13E−02 | 4.42E−02 |
| 34 | C12orf49 | 7.61E−05 | 2.82E−02 |
| 35 | C20orf24 | 1.17E−02 | 3.08E−02 |
| 36 | C3orf14 | 1.03E−02 | 2.70E−04 |
| 37 | CAPNS1 | 1.59E−02 | 1.50E−02 |
| 38 | CAPRIN1 | 4.44E−05 | 1.16E−02 |
| 39 | CCAR1 | 2.02E−02 | 4.63E−02 |
| 40 | CCDC114 | 1.90E−02 | 3.22E−02 |
| 41 | CCDC146 | 2.46E−06 | 2.44E−04 |
| 42 | CCDC6 | 8.24E−04 | 3.96E−02 |
| 43 | CCDC65 | 2.51E−02 | 2.34E−03 |
| 44 | CD58 | 8.66E−03 | 3.21E−02 |
| 45 | CDC42 | 1.77E−03 | 4.97E−02 |
| 46 | CDK14 | 6.27E−05 | 7.92E−04 |
| 47 | CDK4 | 9.89E−03 | 2.78E−03 |
| 48 | CEP192 | 8.79E−03 | 2.84E−02 |
| 49 | CHMP2B | 1.98E−03 | 4.55E−02 |
| 50 | CHMP4B | 5.87E−04 | 4.13E−02 |
| 51 | CLNS1A | 2.20E−03 | 7.24E−03 |
| 52 | CLTA | 2.43E−03 | 1.95E−03 |
| 53 | CNIH1 | 4.72E−02 | 3.93E−02 |
| 54 | COPS3 | 1.72E−02 | 2.20E−03 |
| 55 | CRKL | 1.87E−03 | 2.38E−02 |
| 56 | CSAD | 9.34E−04 | 4.75E−02 |
| 57 | CSE1L | 7.75E−03 | 4.24E−02 |
| 58 | CSNK2A2 | 1.06E−03 | 2.01E−02 |
| 59 | CUL1 | 1.04E−02 | 4.55E−02 |
| 60 | CYB5B | 3.96E−05 | 4.98E−02 |
| 61 | CYP19A1 | 3.52E−02 | 1.94E−02 |
| 62 | CYP20A1 | 4.61E−05 | 1.07E−02 |
| 63 | CYP2E1 | 2.10E−03 | 1.85E−02 |
| 64 | DCTN6 | 4.62E−03 | 3.58E−02 |
| 65 | DDHD1 | 4.19E−02 | 2.73E−03 |
| 66 | DDX1 | 2.35E−03 | 4.13E−02 |
| 67 | DERA | 2.28E−02 | 2.46E−03 |
| 68 | DESI2 | 3.05E−04 | 3.95E−02 |
| 69 | DMAC2 | 4.73E−03 | 1.45E−03 |
| 70 | DNAJB5 | 1.06E−02 | 3.50E−02 |
| 71 | DNAJC1 | 2.30E−03 | 2.85E−02 |
| 72 | DNASE2 | 1.07E−02 | 1.38E−02 |
| 73 | DOCK3 | 4.97E−02 | 4.86E−02 |
| 74 | EAPP | 1.43E−02 | 3.95E−02 |
| 75 | ECH1 | 7.14E−04 | 3.95E−02 |
| 76 | ECHDC2 | 4.65E−04 | 4.59E−02 |
| 77 | EDEM3 | 3.12E−02 | 2.62E−02 |
| 78 | EEF1B2 | 1.06E−04 | 2.21E−02 |
| 79 | EHD2 | 3.84E−05 | 2.07E−02 |
| 80 | EIF2AK2 | 3.74E−05 | 2.39E−02 |
| 81 | EIF2B3 | 1.82E−02 | 4.39E−02 |
| 82 | EIF2S2P4 | 8.92E−03 | 4.07E−03 |
| 83 | EIF3E | 2.96E−06 | 4.92E−02 |
| 84 | EIF3I | 2.32E−05 | 3.55E−02 |
| 85 | EIF4G2 | 2.44E−02 | 3.21E−02 |
| 86 | ELOB | 4.55E−04 | 1.32E−02 |
| 87 | EML2 | 5.41E−03 | 2.37E−02 |
| 88 | EPB41L3 | 8.58E−04 | 4.53E−02 |
| 89 | ERH | 1.75E−05 | 3.50E−03 |
| 90 | ERI1 | 6.23E−05 | 4.43E−02 |
| 91 | ERICH1 | 2.69E−02 | 4.36E−02 |
| 92 | EXOC1 | 2.82E−03 | 4.05E−02 |
| 93 | EXOC4 | 1.91E−03 | 3.91E−03 |
| 94 | FAM160A2 | 3.69E−05 | 2.39E−03 |
| 95 | FAM71F1 | 2.65E−02 | 2.04E−02 |
| 96 | FAM8A1 | 1.76E−04 | 3.94E−02 |
| 97 | FBL | 5.86E−04 | 3.48E−02 |
| 98 | FBXL8 | 5.21E−04 | 2.24E−02 |
| 99 | FBXO9 | 1.73E−02 | 4.21E−03 |
| 100 | FDX1 | 6.84E−05 | 4.10E−03 |
| 101 | FER1L4 | 2.46E−02 | 4.64E−02 |
| 102 | FGF7 | 7.15E−03 | 4.28E−02 |
| 103 | FRG1 | 4.36E−04 | 4.32E−02 |
| 104 | FUCA2 | 9.44E−05 | 3.40E−02 |
| 105 | FUT8 | 7.94E−03 | 1.84E−02 |
| 106 | GBE1 | 5.60E−04 | 3.44E−02 |
| 107 | GDE1 | 1.00E−04 | 1.68E−02 |
| 108 | GIMAP2 | 1.42E−02 | 4.27E−02 |
| 109 | GINM1 | 2.45E−03 | 3.70E−02 |
| 110 | GLUL | 2.28E−03 | 8.91E−03 |
| 111 | GOLGA5 | 3.99E−04 | 3.16E−03 |
| 112 | GOLPH3 | 1.31E−04 | 9.79E−03 |
| 113 | GPNMB | 2.61E−02 | 3.06E−02 |
| 114 | GTPBP10 | 6.60E−04 | 7.96E−03 |
| 115 | GUCD1 | 9.65E−04 | 2.85E−02 |
| 116 | HACD3 | 2.06E−02 | 3.43E−02 |
| 117 | HADHA | 3.06E−02 | 4.04E−02 |
| 118 | HAUS2 | 7.22E−04 | 1.15E−02 |
| 119 | HBP1 | 8.17E−04 | 4.56E−03 |
| 120 | HEMK1 | 1.55E−03 | 4.40E−02 |
| 121 | HGFAC | 4.61E−03 | 4.10E−02 |
| 122 | HNRNPC | 1.47E−04 | 3.47E−02 |
| 123 | HNRNPM | 2.00E−02 | 1.75E−02 |
| 124 | HNRNPUL1 | 4.17E−04 | 2.26E−02 |
| 125 | HOOK2 | 6.32E−04 | 3.94E−02 |
| 126 | HOXA11 | 9.67E−04 | 3.35E−02 |
| 127 | HOXC8 | 1.09E−03 | 3.15E−02 |
| 128 | HOXD3 | 1.99E−03 | 2.56E−02 |
| 129 | HSP90AA1 | 1.11E−03 | 4.29E−02 |
| 130 | HTATSF1 | 9.69E−03 | 3.51E−03 |
| 131 | IARS2 | 2.48E−02 | 4.62E−02 |
| 132 | ICAM1 | 3.19E−02 | 3.08E−02 |
| 133 | ICMT | 4.19E−03 | 8.11E−03 |
| 134 | IK | 1.41E−02 | 3.75E−02 |
| 135 | IL1R1 | 3.92E−03 | 2.75E−03 |
| 136 | IL6ST | 1.43E−03 | 4.09E−02 |
| 137 | JDP2 | 2.68E−02 | 7.30E−03 |
| 138 | KCNK2 | 2.27E−03 | 1.16E−04 |
| 139 | KCTD1 | 6.62E−06 | 1.36E−02 |
| 140 | KIAA1468 | 1.89E−02 | 2.95E−02 |
| 141 | LAMTOR5 | 8.82E−04 | 3.72E−02 |
| 142 | LAP3 | 2.94E−02 | 3.90E−03 |
| 143 | LAPTM4B | 1.64E−02 | 1.18E−02 |
| 144 | LRRC1 | 3.91E−03 | 2.36E−02 |
| 145 | LRRC32 | 3.41E−02 | 4.93E−03 |
| 146 | MAN1A1 | 9.85E−03 | 2.61E−02 |
| 147 | MAP1LC3B | 2.33E−05 | 4.30E−02 |
| 148 | MAPK1 | 3.18E−04 | 2.99E−02 |
| 149 | MAPKAP1 | 1.51E−03 | 6.43E−03 |
| 150 | MAX | 2.62E−02 | 9.70E−03 |
| 151 | MED4 | 4.39E−04 | 3.60E−02 |
| 152 | MFAP1 | 4.14E−02 | 3.95E−03 |
| 153 | MICAL1 | 1.13E−02 | 3.27E−02 |
| 154 | MRM2 | 1.01E−03 | 1.26E−02 |
| 155 | MRO | 2.60E−04 | 3.98E−03 |
| 156 | MRPL3 | 6.54E−04 | 2.97E−02 |
| 157 | MRPS15 | 3.63E−06 | 4.25E−02 |
| 158 | MRPS35 | 7.52E−03 | 3.72E−02 |
| 159 | MSH3 | 1.63E−05 | 2.00E−03 |
| 160 | MTPN | 3.87E−04 | 1.25E−02 |
| 161 | NAA50 | 2.03E−03 | 3.88E−02 |
| 162 | NAMPT | 4.20E−03 | 3.18E−02 |
| 163 | NCALD | 4.79E−02 | 1.27E−02 |
| 164 | NCBP1 | 4.18E−02 | 4.87E−02 |
| 165 | NCL | 2.31E−02 | 1.34E−02 |
| 166 | NDFIP1 | 8.40E−06 | 9.05E−03 |
| 167 | NDUFA8 | 1.49E−03 | 1.47E−02 |
| 168 | NDUFS1 | 2.08E−02 | 1.06E−02 |
| 169 | NINL | 8.97E−04 | 3.97E−02 |
| 170 | NOL10 | 2.66E−02 | 6.16E−03 |
| 171 | NUCKS1 | 1.50E−04 | 2.57E−03 |
| 172 | NUDT15 | 1.18E−04 | 8.12E−03 |
| 173 | NUFIP2 | 3.64E−02 | 3.66E−02 |
| 174 | OGFRL1 | 8.48E−05 | 2.19E−02 |
| 175 | OLFML3 | 2.52E−02 | 7.71E−03 |
| 176 | OMD | 4.12E−02 | 2.69E−03 |
| 177 | OSTF1 | 2.08E−04 | 3.00E−02 |
| 178 | PARK7 | 1.11E−03 | 9.71E−03 |
| 179 | PARP3 | 5.65E−03 | 4.87E−02 |
| 180 | PCNA | 5.00E−04 | 3.98E−03 |
| 181 | PDE4C | 4.49E−02 | 2.91E−02 |
| 182 | PDE8A | 3.73E−02 | 3.86E−02 |
| 183 | PHF5A | 9.88E−04 | 4.14E−02 |
| 184 | PHKA2 | 9.25E−04 | 4.59E−02 |
| 185 | POLA1 | 4.33E−03 | 3.38E−02 |
| 186 | POLR1E | 5.93E−03 | 2.14E−02 |
| 187 | PPM1G | 1.07E−02 | 4.36E−02 |
| 188 | PPP3CA | 5.33E−03 | 5.42E−03 |
| 189 | PRCP | 5.97E−03 | 9.55E−03 |
| 190 | PRDX5 | 8.75E−06 | 9.88E−03 |
| 191 | PRELID3B | 2.07E−04 | 4.22E−02 |
| 192 | PRKAR2A | 6.46E−03 | 2.82E−02 |
| 193 | PRKAR2B | 3.37E−03 | 2.18E−02 |
| 194 | PRPS2 | 2.74E−04 | 6.87E−03 |
| 195 | PSMA4 | 1.38E−02 | 2.65E−02 |
| 196 | PSMA6 | 8.45E−04 | 5.22E−03 |
| 197 | PSMB7 | 2.65E−06 | 6.28E−03 |
| 198 | PSMC1 | 3.34E−02 | 3.42E−02 |
| 199 | PSMD10 | 2.00E−06 | 8.74E−03 |
| 200 | PSMG2 | 3.96E−05 | 3.65E−02 |
| 201 | PYGL | 5.81E−03 | 9.89E−03 |
| 202 | QRSL1 | 7.24E−03 | 9.83E−03 |
| 203 | RAB11A | 4.74E−05 | 2.11E−02 |
| 204 | RAB22A | 9.93E−05 | 1.48E−02 |
| 205 | RAB7A | 6.78E−05 | 5.44E−03 |
| 206 | RALBP1 | 5.12E−03 | 2.80E−02 |
| 207 | RANBP6 | 1.24E−02 | 3.49E−02 |
| 208 | RASA4 | 1.86E−02 | 2.40E−02 |
| 209 | RNF113A | 1.46E−05 | 1.22E−02 |
| 210 | ROMO1 | 1.53E−02 | 4.21E−02 |
| 211 | RP2 | 1.09E−04 | 1.12E−02 |
| 212 | RPGRIP1L | 2.38E−02 | 3.66E−02 |
| 213 | RPL18A | 8.79E−06 | 4.89E−02 |
| 214 | RPL19 | 2.65E−05 | 1.55E−02 |
| 215 | RPL22 | 1.10E−05 | 1.65E−02 |
| 216 | RPL24 | 2.46E−05 | 1.33E−02 |
| 217 | RPL27 | 4.97E−03 | 3.09E−02 |
| 218 | RPL31 | 4.91E−05 | 1.76E−02 |
| 219 | RPL34 | 1.11E−05 | 2.06E−02 |
| 220 | RPL35 | 2.46E−06 | 2.18E−02 |
| 221 | RPL5 | 5.81E−07 | 1.27E−02 |
| 222 | RPS10 | 9.28E−04 | 2.67E−03 |
| 223 | RPS12 | 2.13E−03 | 2.73E−03 |
| 224 | RPS13 | 9.36E−05 | 6.52E−03 |
| 225 | RPS20 | 2.42E−03 | 2.69E−02 |
| 226 | RPS25 | 1.55E−03 | 3.63E−02 |
| 227 | RPS6 | 6.63E−07 | 1.53E−03 |
| 228 | RRAGC | 9.09E−05 | 3.51E−02 |
| 229 | RRP36 | 2.58E−03 | 9.89E−03 |
| 230 | RSL24D1 | 7.83E−04 | 2.26E−02 |
| 231 | RTN3 | 6.00E−04 | 1.06E−02 |
| 232 | SARAF | 1.86E−02 | 2.60E−03 |
| 233 | SCRN1 | 9.33E−04 | 2.90E−03 |
| 234 | SDHB | 1.10E−02 | 4.07E−02 |
| 235 | SEH1L | 4.17E−03 | 4.26E−02 |
| 236 | SERINC1 | 2.30E−03 | 9.64E−03 |
| 237 | SERINC3 | 8.05E−05 | 7.58E−04 |
| 238 | SET | 6.47E−04 | 6.63E−03 |
| 239 | SF3A1 | 2.13E−02 | 3.64E−02 |
| 240 | SF3B1 | 5.38E−06 | 3.94E−03 |
| 241 | SGPP1 | 1.66E−05 | 3.69E−02 |
| 242 | SKAP2 | 1.31E−02 | 3.64E−02 |
| 243 | SLC17A5 | 4.10E−02 | 3.67E−02 |
| 244 | SLC25A1 | 1.67E−02 | 5.94E−03 |
| 245 | SLC31A2 | 9.74E−03 | 1.56E−05 |
| 246 | SLC9A5 | 1.87E−03 | 1.89E−02 |
| 247 | SLF2 | 4.46E−03 | 4.43E−02 |
| 248 | SMAD9 | 1.94E−02 | 4.57E−02 |
| 249 | SMC2 | 7.22E−03 | 4.87E−03 |
| 250 | SMS | 5.19E−04 | 7.28E−03 |
| 251 | SNRPB2 | 2.32E−04 | 2.76E−02 |
| 252 | SNRPF | 1.96E−03 | 4.96E−02 |
| 253 | SNW1 | 2.17E−02 | 2.31E−02 |
| 254 | SNX6 | 1.41E−02 | 4.52E−02 |
| 255 | SNX8 | 1.41E−02 | 2.39E−02 |
| 256 | SOX6 | 2.04E−02 | 2.04E−02 |
| 257 | SPA17 | 2.88E−03 | 4.12E−02 |
| 258 | SPCS2 | 4.82E−04 | 4.60E−02 |
| 259 | SQLE | 2.40E−06 | 4.27E−02 |
| 260 | SRI | 2.15E−02 | 3.60E−02 |
| 261 | STAU1 | 6.40E−04 | 3.47E−02 |
| 262 | SUPT7L | 4.59E−03 | 8.10E−03 |
| 263 | TACO1 | 1.21E−02 | 1.09E−02 |
| 264 | TAF12 | 2.82E−03 | 1.50E−02 |
| 265 | TAF8 | 1.84E−03 | 4.47E−03 |
| 266 | TAX1BP1 | 4.69E−03 | 3.79E−02 |
| 267 | TBC1D9 | 1.77E−03 | 1.23E−02 |
| 268 | TFPI | 2.22E−02 | 4.33E−02 |
| 269 | TGM1 | 5.91E−03 | 7.94E−03 |
| 270 | THAP10 | 4.26E−03 | 1.97E−02 |
| 271 | THG1L | 2.54E−03 | 8.18E−03 |
| 272 | TIMP2 | 3.90E−03 | 2.95E−03 |
| 273 | TMEM14C | 9.85E−03 | 2.50E−02 |
| 274 | TMEM19 | 2.26E−04 | 7.78E−03 |
| 275 | TMEM30A | 3.46E−03 | 4.66E−02 |
| 276 | TMEM54 | 3.80E−03 | 1.04E−04 |
| 277 | TMPO | 2.62E−02 | 2.98E−03 |
| 278 | TNFRSF19 | 6.73E−03 | 2.11E−02 |
| 279 | TPT1 | 8.28E−03 | 3.04E−03 |
| 280 | TRAM1 | 2.91E−03 | 4.06E−02 |
| 281 | TRIM24 | 2.95E−06 | 4.03E−02 |
| 282 | TRIM35 | 1.46E−02 | 1.85E−02 |
| 283 | TSNAXIP1 | 5.94E−03 | 3.39E−02 |
| 284 | TTC17 | 1.06E−04 | 1.83E−02 |
| 285 | TUSC3 | 2.95E−02 | 1.29E−02 |
| 286 | TXLNG | 4.91E−02 | 4.56E−02 |
| 287 | TXN2 | 4.14E−04 | 1.16E−02 |
| 288 | TXNL4B | 1.53E−02 | 4.38E−02 |
| 289 | UBE2G1 | 4.25E−03 | 7.21E−03 |
| 290 | UBE2R2 | 8.94E−04 | 4.63E−02 |
| 291 | UCHL5 | 6.11E−03 | 1.14E−02 |
| 292 | UQCC2 | 3.53E−03 | 1.58E−02 |
| 293 | USP35 | 2.29E−04 | 2.25E−02 |
| 294 | USP8 | 2.96E−02 | 1.24E−03 |
| 295 | VDR | 2.90E−02 | 4.16E−02 |
| 296 | VPS25 | 1.71E−02 | 1.44E−02 |
| 297 | VPS26A | 2.23E−04 | 7.27E−03 |
| 298 | VPS35 | 9.43E−03 | 4.62E−03 |
| 299 | WASL | 4.29E−03 | 1.37E−02 |
| 300 | WBP11 | 3.60E−03 | 2.32E−02 |
| 301 | WIPI1 | 6.07E−03 | 2.99E−02 |
| 302 | WISP2 | 1.67E−02 | 6.00E−04 |
| 303 | XPNPEP2 | 1.67E−02 | 2.40E−06 |
| 304 | YBX3 | 1.52E−02 | 6.04E−03 |
| 305 | YY1 | 1.20E−02 | 1.69E−02 |
| 306 | ZC3HAV1 | 4.36E−03 | 9.83E−04 |
| 307 | ZCCHC6 | 4.55E−02 | 2.00E−02 |
| 308 | ZNF211 | 8.64E−04 | 2.04E−02 |
| 309 | ZNF227 | 2.64E−02 | 2.79E−02 |
| 310 | ZNF337 | 1.11E−03 | 4.74E−02 |
| 311 | ZZEF1 | 7.78E−03 | 4.55E−02 |

TABLE 12
Common dysregulated genes for AD/NDC and AD/Non-
ADD under a statistical significance of P < 0.05.

| AD vs Non-ADD | | | | AD vs NDC | | |
|---|---|---|---|---|---|---|
| | | Training | | | Training | Validation |
| # | Gene name | Set | Validation | Gene name | Set | Set |
| 1 | AC004057.1 | 0.0246 | 0.0199 | AC004057.1 | 0.0048 | 0.0039 |
| 2 | ACP6 | 0.0332 | 0.0169 | ACP6 | 0.0173 | 0.0055 |
| 3 | ADAM20 | 0.0321 | 0.0082 | ADAM20 | 0.0317 | 0.0208 |
| 4 | RPL5 | 0.022 | 0.0422 | RPL5 | 0.0000 | 0.0127 |
| 5 | SHISA5 | 0.0188 | 0.0169 | SHISA5 | 0.0219 | 0.0462 |
| 6 | SNHG14 | 0.0292 | 0.0259 | SNHG14 | 0.0045 | 0.0071 |
| 7 | WASF2 | 0.0027 | 0.0476 | WASF2 | 0.0013 | 0.0196 |
| 8 | ZNF444 | 0.0158 | 0.0056 | ZNF444 | 0.0002 | 0.0145 |

### REFERENCES

1. Chen M et al. “Serum Starvation Induced Cell Cycle Synchronization Facilitates Human Somatic Cells Reprogramming”, PLoS ONE 7(4) (2012).
2. Baghdadchi N. “The Effects of Serum Starvation on Cell Cycle Synchronization”, OSR Journal of Student Research (2013).
3. Hayes 0 et al. “Cell confluency is as efficient as serum starvation for inducing arrest in the G0/G1 phase of the cell cycle in granulosa and fibroblast cells of cattle”, Anim. Reprod. Sci. 87(3-4):181-92 (2005).
4. Spellman PT et al, “Comprehensive identification of cell cycle-regulated genes of the yeast Saccharomyces cerevisiae by microarray hybridization” Mol. Biol. Cell. 9(12):3273-97(1998).

1.-51. (canceled)

52. A composition of matter comprising a population of synchronized cultured human skin cell fibroblasts, wherein at least 95% of the cells in the population are synchronized.

53. A composition of matter comprising a population of synchronized cultured skin cell fibroblasts from a human subject suspected of having dementia, wherein at least 95% of the cells in the population are synchronized.

54. A composition of matter comprising a population of synchronized cultured skin cell fibroblasts from a human subject suspected of having dementia, wherein (i) at least 95% of the cells in the population are synchronized, and (ii) the expression levels in the cell population of at least one of genes AC004057.1, AC092651.1, ACP6, ADAM20, ASXL2, C2CD5, CARNS1, FAM149B1, GLIS3-AS1, IL18R1, LINC01393, LZIC, MAP1LC3B2, NHLH1, NORAD, NPPA-AS1_3, OSMR-AS1, PAN3, PHBP8, PSMB9, RAB3IP, RDH16, RFESDP1, RPL5, SCG2, SDHD, SHISA5, SLC45A3, SNHG14, TTC26, URB2, USMG5, WASF2, ZCWPW2, ZNF444, and ZNF70, when measured as the number of RNA transcripts per million RNA transcripts, are consistent with the genes' expression levels in corresponding synchronized cells derived from Alzheimer's disease patients.

55. The composition of claim 52, wherein the expression level of AC004057.1 in the cell population is greater than 161.81 RNA transcripts per million RNA transcripts.

56. The composition of claim 52, wherein the expression level of ACP6 in the cell population is less than 2.84 RNA transcripts per million RNA transcripts.

57. The composition of claim 52, wherein the expression level of ADAM20 in the cell population is less than 0.16 RNA transcripts per million RNA transcripts.

58. The composition of claim 52, wherein the expression level of ASXL2 in the cell population is less than 0.76 RNA transcripts per million RNA transcripts.

59. The composition of claim 52, wherein the expression level of C2CD5 in the cell population is greater than 28.76 RNA transcripts per million RNA transcripts.

60. The composition of claim 52, wherein the expression level of CARNS1 in the cell population is greater than 0.15 RNA transcripts per million RNA transcripts.

61. The composition of claim 52, wherein the expression level of FAM149B1 in the cell population is less than 22.60 RNA transcripts per million RNA transcripts.

62. The composition of claim 52, wherein the expression level of LINC01393 in the cell population is greater than 0.86 RNA transcripts per million RNA transcripts.

63. The composition of claim 52, wherein the expression level of MAP1LC3B2 in the cell population is greater than 4.41 RNA transcripts per million RNA transcripts.

64. The composition of claim 52, wherein the expression level of NHLH1 in the cell population is less than 0.27 RNA transcripts per million RNA transcripts.

65. The composition of claim 52, wherein the expression level of NPPA-AS 1_3 in the cell population is less than 2.54 RNA transcripts per million RNA transcripts.

66. The composition of claim 52, wherein the expression level of PAN3 in the cell population is less than 15.98 RNA transcripts per million RNA transcripts.

67. The composition of claim 52, wherein the expression level of PHBP8 in the cell population is greater than 0.98 RNA transcripts per million RNA transcripts.

68. The composition of claim 52, wherein the expression level of PSMB9 in the cell population is greater than 18.00 RNA transcripts per million RNA transcripts.

69. The composition of claim 52, wherein the expression level of RAB3IP in the cell population is less than 0.50 RNA transcripts per million RNA transcripts.

70. The composition of claim 52, wherein the expression level of RPL5 in the cell population is greater than 794.88 RNA transcripts per million RNA transcripts.

71. The composition of claim 52, wherein the expression level of SCG2 in the cell population is less than 0.68 RNA transcripts per million RNA transcripts.

72. The composition of claim 52, wherein the expression level of SHISA5 in the cell population is greater than 107.32 RNA transcripts per million RNA transcripts.

73. The composition of claim 52, wherein the expression level of SLC45A3 in the cell population is less than 1.11 RNA transcripts per million RNA transcripts.

74. The composition of claim 52, wherein the expression level of TTC26 in the cell population is greater than 2.41 RNA transcripts per million RNA transcripts.

75. The composition of claim 52, wherein the expression level of URB2 in the cell population is greater than 2.28 RNA transcripts per million RNA transcripts.

76. The composition of claim 52, wherein the expression level of USMG5 in the cell population is greater than 129.13 RNA transcripts per million RNA transcripts.

77. The composition of claim 52, wherein the expression level of WASF2 in the cell population is greater than 23.74 RNA transcripts per million RNA transcripts.

78. The composition of claim 52, wherein the expression level of ZCWPW2 in the cell population is greater than 1.15 RNA transcripts per million RNA transcripts.

79. The composition of claim 52, wherein the expression level of ZNF444 in the cell population is less than 16.73 RNA transcripts per million RNA transcripts.

80. The composition of claim 52, wherein the expression level of ZNF70 in the cell population is greater than 0.87 RNA transcripts per million RNA transcripts.
